(19) United States
(12) Patent Application Publication
(10) Pub. No.: US 2023/0172971 A1
(43) Pub. Date: Jun. 8, 2023

(54) ANTIBIOTIC AND ANTI-INFLAMMATORY COMPOSITIONS AND METHODS OF USE

(71) Applicant: The Board of Regents of the University of Oklahoma, Norman, OK (US)

(72) Inventor: CHARLES V. RICE, Norman, OK (US)

(21) Appl. No.: 17/922,674

(22) PCT Filed: Apr. 29, 2021
(86) PCT No.: PCT/US21/70485

§ 371 Date: Nov. 1, 2022

Related U.S. Application Data
(60) Provisional application No. 63/018,693, filed on May 1, 2020.
(60) Provisional application No. 63/020,600, filed on May 6, 2020.
(60) Provisional application No. 63/123,115, filed on Dec. 9, 2020.

(51) Int. Cl. A61K 31/785; A61K 47/60; A61P 29/00; A61P 31/04; A61K 31/7048; A61K 31/7052; A61P 17/02

(52) U.S. Cl. CPC A61K 31/785; A61K 47/60; A61P 29/00; A61P 31/04; A61K 31/7048; A61K 31/7052; A61P 17/02

(57) ABSTRACT

Compositions, kits, and methods of use of branched poly(ethylenimine) (BPEI) compounds, including polyethylene glycol (PEG)-branched poly(ethylenimine) conjugates (PEG-BPEI) for treatment of diseases, bacterial infections, bacterial biofilms, and conditions associated with inflammation, including wounds.

### CROSS REFERENCE TO RELATED APPLICATIONS

The present patent application claims priority under 35 U.S.C. 119(e) to U.S. Provisional patent application U.S. Ser. No. 63/018,693, filed on May 1, 2020, U.S. Provisional patent application U.S. Ser. No. 63/020,600, filed on May 6, 2020 and U.S. Provisional patent application U.S. Ser. No. 63/123,115, filed on Dec. 9, 2020, and incorporates herein by reference the entire contents of each of said applications.

### GOVERNMENT SUPPORT

This invention was made with government support under Grant Number R03 AI142420-02 awarded by the National Institutes of Health (NIH). The government has certain rights in the invention.

### BACKGROUND

The innate immune system is one of the first-line defense mechanisms in the human body, especially in wound healing. As one of the most complex biological events, wound healing involves many interactive processes mediated by local resident cells (keratinocytes, fibroblasts, nerve cells) and infiltration of neutrophils, mast cells, macrophages, and lymphocytes to trigger production of growth factors and cytokines. Cytokines are chemical messengers produced by all immune cells for their communication. Interleukins (ILs) are those serve for leukocyte communication. The activation and suppression of immune system or cell division are closely related to interleukins. For example, interleukin IL-6 functions in proliferative phase of healing. IL-6 was found with significantly elevated amount in chronic wounds compared to acute wounds, suggesting the inflammatory phase is likely stuck in chronic wound healing process. Chemokines are a group of cytokines that are responsible for leukocyte recruitment into sites of infection or injury and for maintenance of inflammatory reaction, with the best known is IL-8 (or CXCL-8). Peak levels of IL-8 are often by neutrophils and found under a wound surface. Another subgroup of cytokines is tumor necrosis factors (TNFs), which mainly activate immune cells to sites of infection and to tumor cells to destroy them. TNFs are cytotoxic specifically to tumor cells, with TNF-α is the most outstanding member in having many physiologic functions including the survival and death of other cells. Expression of proinflammatory cytokine TNF-α also induces the production of other cytokines (e.g., IL-1, IL-6, and IL-8) that increase the inflammatory responses of leukocytes.

Inflammation is a natural response of the immune system and is critical to eliminating the infectious agent and coordinating the delivery of defenses to the infection site. There are different ways inflammation can be triggered, including by bacterial endotoxins, also known as lipopolysaccharide (LPS). Pro-inflammatory responses of the immune system are generally good for the host. However, these responses must be counterbalanced by anti-inflammatory mediators to prevent harming the host.

LPS released from Gram-negative bacteria causes the production of inflammatory cytokines including tumor necrosis factor alpha (TNFα), interleukin-6 (IL-6), interleukin-1β, and Nitric Oxide (NO). Cytokines are involved in every aspect of the immune response and inflammation. The type of cytokine secreted determines whether an immune response is necessary and whether that response is cytotoxic, humoral, cell-mediated, or allergic. The cellular receptor that primarily recognizes LPS is the Toll-like receptor 4 (TLR4). TLR4 activation requires interaction with a number of co-receptors including LPS-binding protein (LBP), CD14, and myeloid differentiation protein 2 (MD-2) to bind LPS and induce a signaling cascade. Ultimately, this leads to the activation of NF-κB and the production of pro-inflammatory cytokines.

Excessive production of cytokines caused by LPS-release can lead to toxic shock and sepsis. Once the body fails to regulate its own immune response, normal blood processes can be overwhelmed. In addition, delays in healing acute skin and soft tissue infections are often due to a prolonged inflammatory phase of healing caused by bacterial debris (e.g., LPS). Consequently, preventing LPS from triggering the release of inflammatory cytokines can restore a more balanced inflammatory response. Successful drugs are elusive however because the debris originates from many different species of Gram-negative bacteria. Therefore, the need exists for a broad-spectrum therapeutic agent which can inhibit excess cytokine release. Such an agent could be used to reduce disturbances to the immunologic equilibrium, thereby preventing many acute wounds from becoming chronic wounds and reducing the risk of recurrent infection and tissue necrosis.

The World Health Organization has developed the global priority pathogens list of antibiotic-resistant bacteria. Included in the list of “Priority 1: Critical” pathogens are carbapenem-resistant Enterobacteriaceae (CRE). In this class, dangerous Escherichia coli and Klebsiella pneumoniae strains are resistant to nearly all available antibiotics. The lack of effective and timely treatments leads to severe illness and, in some cases, death. There is a high mortality rate and survivors can have severe morbidity from treatment with toxic last-resort antibiotics. The danger from CRE arises because these bacteria produce enzymes that degrade a wide range of antibiotics, including carbapenems and β-lactams. The predominant carbapenemases are the Klebsiella pneumoniae carbapenemases (KPCs). A greater concern are metallo-β-lactamases (MBLs) because they degrade more antibiotics than KPCs.

In recent years, a variety of new compounds have emerged to combat CRE infections. CRE possess extended-spectrum β-lactamase enzymes and recommended agents against CRE are combinations of an antibiotic and β-lactamase inhibitor (such as ceftazidime-avibactam, meropenem-vaborbactam, imipenem-relebactam, or ceftaroline-avibactam), or a monotherapy of cefiderocol, tigecycline, eravacycline, plazomycin, or gentamycin. Colistin and related polymyxins are recommended as last-resort antibiotics due to toxicity that can cause patient morbidity. The presence of KPCs create complicated treatment decisions as the agents listed above may have antibiotic efficacy reduced or eliminated. There are even fewer effective agents against the more dangerous CRE that express MBLs, such as the New-Delhi MBL (NDM-1).

Although CRE is more commonly associated with urinary tract infections, their presence of wound and skin and soft-tissue infections (SSTIs) cannot be overlooked. A 2018 study reported that 24% (2,521) of 10,698 non-susceptible isolates were from skin/wound infections and treatment options are similar to those listed above. A 2017 analysis of the CRACKLE-1 surveillance data revealed the importance of wounds in CRE transmission. The 4.5 million chronic wound infections increase both the risk of CRE infection and of transferring resistance genes to other species. Treating chronic wounds requires repeated visits to, and in-patient stays in, healthcare facilities which increases the likelihood of spreading CRE. Cefiderocol is an alternative treatment option for CRE infections, regardless of the carbapenem resistance mechanism. But this agent, along with the other mono- and combination agents listed above, are unable to disable biofilms that perpetuate wound infections. There is clearly an unmet need for additional, more effective treatments for infections, such as infected wounds, that are associated with CRE.

Experts predict that by 2050, antimicrobial resistance (AMR) will be the leading cause of death, claiming 10 million lives a year, exceeding the number of deaths caused by cancer today. A swift global response is required to prevent this alarming scenario, but pharmaceutical companies are facing significant market pressures that hinder their ability to meet this need. The cost of bringing a drug to market is extraordinary, up to a billion dollars, yet there are little or no incentives for clinicians to use the new drug. New antibiotics are held in reserve as drugs of last resort to prevent the emergence of resistance. Instead, the paradigm of antibiotic potentiators has emerged to overcome resistance barriers and restore efficacy to existing antibiotics; thereby providing an opportunity to kill drug-resistant and drug-susceptible bacteria with the same formulation. However, antibiotic+potentiator combinations are being developed against Gram-negative pathogens or Gram-positive pathogens rather than a broad-spectrum formulation against both.

Crossing the bacterial membrane is a difficult task for many antimicrobial drugs that must reach intracellular targets of Gram-positive and Gram-negative bacteria in order to have an antibiotic effect. Improving antibiotic efficacy can be accomplished with potentiation adjuvants comprising a vast array of different compounds and targets. A common mechanism for doing so is weakening the cell envelope framework. The outermost portions of the cell envelope of Gram-negative and Gram-positive bacterial pathogens are under exploited weaknesses in antimicrobial resistance mechanisms. Many efforts are focused on inhibitors to the cytoplasmic expression and/or the membrane translocation of essential proteins, enzymes, and precursors required for the assembly of molecules required for the cell-envelope machinery and architecture. These approaches may suffer from deleterious protein binding effects or have low solubility from hydrophobic properties necessary to cross the membrane barriers. Likewise, methods to overcome resistance are different depending on whether the pathogen is a Gram-positive or Gram-negative bacterium.

The divergent approaches to overcome resistance arise from the intrinsic nature of bacterial cell walls and their differing mechanisms of antibiotic resistance. The cytoplasm of Gram-positive bacterial cells is surrounded by a single phospholipid bilayer and this membrane is surrounded by a thick layer of peptidoglycan interlaced with anionic teichoic acids. However, the phospholipid membrane of Gram-negative bacteria is encased by a periplasm region that contains a thin peptidoglycan layer attached to an asymmetric outer membrane bilayer. The inner leaflet of the outer member contains phospholipids, but the outer leaflet contains lipopolysaccharides. Together, these layers make up a formidable barrier to the influx and/or diffusion of antibiotics into the periplasm and cytoplasm to reach their drug targets. Approaches to disable resistance from β-lactamase enzymes and efflux pumps with inhibitors are often tailored for Gram-negative bacteria, such as multidrug-resistant Pseudomonas aeruginosa (MDR-PA), and rarely work against Gram-positive bacteria that lack these primary resistance mechanisms. Resistance in Gram-positive bacteria, for example methicillin-resistant Staphylococcus aureus (MRSA), is dominated by alternative means to continue the assembly and synthesis of peptidoglycan, thereby bypassing the activity of β-lactams.

First-line antibiotics include the β-lactam class of antibiotics, considered among the safest antibiotics to use. In 2015, the US had 269.3 million antibiotic prescriptions given by healthcare providers, which is equivalent to 838 prescriptions per 1000 people. Among them, β-lactams (e.g., penicillin, oxacillin, and amoxicillin) were the most popular prescribed antibiotics with amoxicillin at the top of the chart at 171 prescriptions per 1000 people. For patients who have penicillin allergies, erythromycin and other broad-spectrum macrolides are prescribed as standard of care antibiotics. Since the 1950s, erythromycin has been widely used as a substitute for β-lactams for penicillin-allergic patients. It is a first-line treatment for many pediatric infections. However, clinical isolates of MRSA do not respond to erythromycin treatment. Because erythromycin targets protein synthesis instead of the cell wall, it could be effective against methicillin-resistant staphylococci if the drug was able to reach the cytoplasm.

### BRIEF DESCRIPTION OF THE DRAWINGS

Several embodiments of the present disclosure are hereby illustrated in the appended drawings. It is to be noted however, that the appended drawings only illustrate several typical embodiments and are therefore not intended to be considered limiting of the scope of the present disclosure.

FIG. 1 shows ELISA assays show the amount of cytokine IL-8 released by human epithelial keratinocytes (HEKa cells) in responses to: peptidoglycan (PGN) and 600-Da BPEI (A); combinations of PGN and 600-Da BPEI (B). Data are shown as average of triplicate trials. Error bars denote standard deviation. Statistical analysis with the student's t-test generates p-values of <0.05% (95% confidence, denoted by *) and <0.01 (99% confidence, denoted by **). nd=no statistical difference.

FIG. 2 shows cytokines released by HEKa cells in response to LPS, PG (peptidoglycan), and various concentrations of 600-Da BPEI: IL-8 (upper left panel), IL-6 (upper right panel), TNF-alpha (lower panel). Data show the average of triplicate trials. Error bars denote standard deviation. (*) indicates significant difference found between that sample and the untreated control (t-test, p-value<0.05). PG and LPS cause significant release of IL-8, IL-6, and TNF-alpha, while all concentrations of BPEI tested do not cause cytokine release.

FIG. 3 shows that 600-Da BPEI reduces release of IL-8 (upper panels) by HEKa cells treated with LPS or PG. 600-Da BPEI reduces release of IL-6 (lower left panel) by HEKa cells treated with PG. 600-Da BPEI reduces release of TNF-alpha (lower right panel) by HEKa cells treated with LPS. Data shown the average of triplicate trials. Error bars denote standard deviation. (*) indicates significant difference found between that sample and the corresponding PG or LPS-treated by itself (t-test, p-value<0.05). BPEI suppresses the cytokine release caused by LPS and PG.

FIG. 4 is a thermogram of ITC data of 600-Da BPEI binding showing that 2.5 BPEI's bind to each LPS molecule in P. aeruginosa (open circles), and one BPEI bind to 2 LPS molecule in E. coli (black diamonds). 600-Da BPEI has weaker interactions with E. coli than observed with P. aeruginosa.

FIG. 5 is an illustration of a possible mechanism of action for BPEI showing that LPS leads to cytokine production when it stimulates TLR-4 (left). However, this process is interrupted when BPEI binds with LPS to prevent its recognition by receptors (right).

FIG. 6 shows that BPEI and PEG-BPEI neutralizes LPS endotoxin.

FIG. 7A shows checkerboard assay results for killing carbapenem-resistant Enterobacteriaceae (CRE) strain Klebsiella pneumoniae BAA-2146 with meropenem (MER) antibiotic. K. pneumoniae BAA-2146 expresses NDM-1 and resistance to MER is disabled with 16 μg/mL of 600 Da BPEI. These results are characterized as synergistic. The MIC for 600 Da BPEI is ≥1024 μg/mL. Shaded blocks represent cell growth and open blocks represent no growth.

FIG. 7B shows checkerboard assay results for killing CRE strain K. pneumoniae BAA-2146 with MER antibiotic. Resistance to MER is disabled with 16 μg/mL of PEG350-BPEI. These results are characterized as synergistic. The MIC for PEG350-BPEI is ≥1024 μg/mL. Shaded blocks represent cell growth and open blocks represent no growth.

FIG. 7C shows checkerboard assay results for killing CRE strain K. pneumoniae BAA-2146 with imipenem (IMI) antibiotic. Resistance to IMI is disabled with 16 μg/mL of 600 Da BPEI. These results are characterized as synergistic. The MIC for 600 Da BPEI is ≥1024 μg/mL. Shaded blocks represent cell growth and open blocks represent no growth.

FIG. 7D shows checkerboard assay results for killing CRE strain K. pneumoniae BAA-2146 with IMI antibiotic. Resistance to IMI is disabled with 16 μg/mL of PEG350-BPEI. These results are characterized as synergistic. The MIC for PEG350-BPEI is ≥1024 μg/mL. Shaded blocks represent cell growth and open blocks represent no growth.

FIG. 7E shows checkerboard assay results for killing CRE strain E. coli BAA-2452 with MER antibiotic. K. pneumoniae BAA-2452 expresses NDM-1 and resistance to MER is disabled with 16 μg/mL of 600 Da BPEI. These results are characterized as synergistic. The MIC for 600 Da BPEI is ≥1024 μg/mL. Shaded blocks represent cell growth and open blocks represent no growth.

FIG. 7F shows checkerboard assay results for killing CRE strain E. coli BAA-2452 with MER antibiotic. Resistance to MER is disabled with 16 μg/mL of PEG350-BPEI. These results are characterized as synergistic. The MIC for PEG350-BPEI is ≥1024 μg/mL. Shaded blocks represent cell growth and open blocks represent no growth.

FIG. 7G shows checkerboard assay results for killing CRE strain E. coli BAA-2452 with IMI antibiotic. Resistance to IMI is disabled with 16 μg/mL of 600 Da BPEI. These results are characterized as synergistic. The MIC for 600 Da BPEI is ≥1024 μg/mL. Shaded blocks represent cell growth and open blocks represent no growth.

FIG. 7H shows checkerboard assay results for killing CRE strain E. coli BAA-2452 with IMI antibiotic. Resistance to IMI is disabled with 16 μg/mL of PEG350-BPEI. These results are characterized as synergistic. The MIC for PEG350-BPEI is ≥1024 μg/mL. Shaded blocks represent cell growth and open blocks represent no growth.

FIG. 8 shows isothermal titration calorimetry data which demonstrate that 600 Da BPEI (closed circles) and PEG350-BPEI (open circles) bind Zn²⁺ ions. The differences in binding energetics and molar ratio are attributed to steric hindrance from the 350 MW PEG group attached to 600 Da BPEI. This effect shows that PEGylation does not influence the ability to chelate Zn²⁺ ions and thus explains why 600 Da BPEI and PEG350-BPEI have similar MER and IMI potentiation effects against CRE that express NDM-1.

FIG. 9A shows fluorescence intensity data for the intracellular accumulation of the DNA-binding compound H33342 in E. coli BAA-2452. Both 600-Da BPEI (squares) and PEG350-BPEI (triangles) increase the uptake of H33342 by E. coli BAA-2452 over untreated cells (open circles). Closed circles represent treatment with polymyxin-B. The treatment agent concentrations are non-lethal because the cell density, 7×10⁸ CFU/mL, is higher than the cell density used in the growth inhibition assays (5×10⁵ CFU/mL). These data are the average of 5 trials. The results for treated cells (squares, triangles, closed circles) are significant at at least the 95% level over untreated cells (open circles).

FIG. 9B shows fluorescence intensity data for the intracellular accumulation of the DNA-binding compound H33342 in K. pneumoniae BAA-2146. Both 600-Da BPEI (squares) and PEG350-BPEI (triangles) increase the uptake of H33342 over untreated cells (open circles) but the effect is lower in K. pneumoniae BAA-2146 than in by E. coli BAA-2452 (FIG. 9A). The treatment agent concentrations are non-lethal because the cell density, 7×10⁸ CFU/mL, is higher than the cell density used in the growth inhibition assays (5×10⁵ CFU/mL). These data are the average of 5 trials. The results for treated cells (squares, triangles, closed circles) are significant at at least the 95% level over untreated cells (open circles) after 5 minutes of treatment time.

FIG. 10A shows checkerboard assay results for the effectiveness of piperacillin (PIP) antibiotic against CRE strain K. pneumoniae BAA-2146 when also treated with 600-Da BPEI. PIP in combination with 600-Da BPEI is essentially ineffective against K. pneumoniae BAA-2146. Shaded blocks represent cell growth and open blocks represent no growth.

FIG. 10B shows checkerboard assay results for the effectiveness of PIP plus the (3-lactamase inhibitor tazobactam (TAZO) against CRE strain K. pneumoniae BAA-2146 when also treated with 600-Da BPEI. PIP and TAZO in combination with 600-Da BPEI are essentially ineffective against K. pneumoniae BAA-2146. Shaded blocks represent cell growth and open blocks represent no growth.

FIG. 10C shows checkerboard assay results for the effectiveness of PIP plus the (3-lactamase inhibitor TAZO against CRE strain K. pneumoniae BAA-2146 when also treated with PEG350-BPEI. PIP and TAZO in combination with PEG350-BPEI are essentially ineffective against K. pneumoniae BAA-2146. Shaded blocks represent cell growth and open blocks represent no growth.

FIG. 10D shows checkerboard assay results for the effectiveness of PIP against CRE strain E. coli BAA-2452 when also treated with 600-Da BPEI. 600-Da BPEI potentiates the effectiveness of PIP against against E. coli BAA-2452. Shaded blocks represent cell growth and open blocks represent no growth.

FIG. 10E shows checkerboard assay results for the effectiveness of PIP together with TAZO against CRE strain E. coli BAA-2452 when also treated with 600-Da BPEI. 600-Da BPEI potentiates the effectiveness of the PIP plus TAZO combination against E. coli BAA-2452. Shaded blocks represent cell growth and open blocks represent no growth.

FIG. 10F shows checkerboard assay results for the effectiveness of PIP together with TAZO against CRE strain E. coli BAA-2452 when also treated with PEG350-BPEI. PEG350-BPEI potentiates the effectiveness of the PIP plus TAZO combination against E. coli BAA-2452. Shaded blocks represent cell growth and open blocks represent no growth.

FIG. 11A shows checkerboard assay results for effectiveness of MER against CRE strain K. pneumoniae BAA-1705(bla_{KPC}_{⁺}/bla_{NDM}_{^{−}}) when also treated with 600-Da BPEI. MER in combination with 600-Da BPEI are not sufficient to reduce the meropenem MIC to values below its resistance breakpoint of 8 μg/mL. Shaded blocks represent cell growth and open blocks represent no growth.

FIG. 11B shows checkerboard assay results for effectiveness of MER in combination with a β-lactamase inhibitor (TAZO) against CRE strain K. pneumoniae BAA-1705(bla_{KPC}_{⁺}/bla_{NDM}_{^{−}}) when also treated with 600-Da BPEI. MER plus TAZO, in combination with 600-Da BPEI, are able to reduce the meropenem MIC to values below its resistance breakpoint of 8 μg/mL. Shaded blocks represent cell growth and open blocks represent no growth.

FIG. 11C shows checkerboard assay results for effectiveness of IMI against CRE strain K. pneumoniae BAA-1705(bla_{KPC}_{⁺}/bla_{NDM}_{^{−}}) when also treated with 600-Da BPEI. IMI in combination with 600-Da BPEI are not sufficient to reduce the imipenem MIC to values below its resistance breakpoint of 2 μg/mL. Shaded blocks represent cell growth and open blocks represent no growth.

FIG. 11D shows checkerboard assay results for effectiveness of IMI in combination with TAZO against CRE strain K. pneumoniae BAA-1705(bla_{KPC}_{⁺}/bla_{NDM}_{^{−}}) when also treated with 600-Da BPEI. MER plus TAZO, in combination with 600-Da BPEI, are not sufficient to reduce the IMI MIC to values below its resistance breakpoint of 2 μg/mL. Shaded blocks represent cell growth and open blocks represent no growth.

FIG. 12A shows checkerboard assay results for effectiveness of MER against CRE strain E. coli BAA-2340(bla_{KPC}_{⁺}/bla_{NDM}_{^{−}}) when also treated with 600-Da BPEI. MER in combination with 600-Da BPEI are not sufficient to reduce the meropenem MIC to values below its resistance breakpoint. Shaded blocks represent cell growth and open blocks represent no growth.

FIG. 12B shows checkerboard assay results for effectiveness of MER in combination with β-lactamase inhibitor TAZO against CRE strain E. coli BAA-2340(bla_{KPC}_{⁺}/bla_{NDM}_{^{−}}) when also treated with 600-Da BPEI. MER plus TAZO, in combination with 600-Da BPEI, are able to reduce the meropenem MIC to values below its resistance breakpoint. Shaded blocks represent cell growth and open blocks represent no growth.

FIG. 12C shows checkerboard assay results for effectiveness of IMI against CRE strain E. coli BAA-2340(bla_{KPC}_{⁺}/bla_{NDM}_{^{−}}) when also treated with 600-Da BPEI. IMI in combination with 600-Da BPEI are not sufficient to reduce the imipenem MIC to values below its resistance breakpoint. Shaded blocks represent cell growth and open blocks represent no growth.

FIG. 12D shows checkerboard assay results for effectiveness of IMI in combination with TAZO against CRE strain E. coli BAA-2340(bla_{KPC}_{⁺}/bla_{NDM}_{^{−}}) when also treated with 600-Da BPEI. IMI plus TAZO, in combination with 600-Da BPEI, are able to reduce the imipenem MIC to values below its resistance breakpoint. Shaded blocks represent cell growth and open blocks represent no growth.

FIG. 13A is an illustration showing the mechanism of action on 600-Da branched polyethylenimine (BPEI) on Gram-positive cell wall and membrane. Cationic BPEI not only binds anionic wall teichoic acid (WTA) to indirectly disable penicillin binding proteins PBP2a/4 (which only function properly by localization of WTA), it also electrostatically binds the phosphate heads of the lipid membrane, causing a partial loss of the permeability barrier.

FIG. 13B is an illustration showing the mechanism of action on 600-Da BPEI on Gram-negative cell wall and membrane. Cationic BPEI binds to LPS in Gram-negative bacteria creating new hydrophilic conduits to enhance drug-influx.

FIG. 14 shows checkerboard data presentation of bacterial growth inhibition from the combination of erythromycin and 600-Da BPEI. The MICs in these assays can be used to show synergy in the clinical isolates: MRSA OU6 (A, FICI=0.26), MRSA OU11 (B, FICI=0.31), and PA OU19 (C, FICI=0.26). Shaded blocks represent cell growth and open blocks represent no growth. Each assay was performed in triplicate and the data presented above are the average of these assays.

FIG. 15 shows H33342 fluorescence permeation curves demonstrating that the addition of BPEI (128 μg/mL) enhances the cell-membrane permeability of MRSA OU6 and OU11 as the fluorescence of H33342 increased, compared to their untreated control. Error bars denote standard deviation (n=5).

FIG. 16 shows H33342 fluorescence permeation curves demonstrating the addition of 128 μg/mL polymyxin B (PmB) drastically increases the dye uptake more than twice of that 128 μg/mL BPEI does on P. aeruginosa PA OU19. Error bars denote standard deviation (n=6).

FIG. 17 shows H33342 fluorescence permeation curves demonstrating the addition of 128 μg/mL polymyxin B (PmB) drastically increases the dye uptake by MRSA OU11, more than twice of that 128 μg/mL BPEI does. Error bars denote standard deviation (n=5).

FIG. 18 shows Resazurin assay data used to evaluate cell viability of MRSA OU11 (at the cell density of ˜7×10⁹ CFU/mL) treated with either BPEI or polymyxin B (PmB). Resazurin is converted to resorufin by cellular metabolism product NADH/H⁺ and thus provide an indication of cell viability. Error bars denote standard deviation (n=8).

FIG. 19 shows ELISA data indicating the amount of cytokine IL-8 released by human epithelial keratinocytes (HEKa cells) in responses to (A) peptidoglycan (PGN) and 600-Da BPEI and (B) combinations of PGN and 600-Da BPEI. Data are shown as average of triplicate trials. Error bars denote standard deviation. Statistical analysis with the student's t-test generates p-values of <0.05% (95% confidence, denoted by *) and <0.01 (99% confidence, denoted by **). nd=no statistical difference.

FIG. 20 shows a checkerboard data presentation of inhibition of growth of K. pneumoniae ATCC 43816 by a combination of erythromycin and 600-Da BPEI. The MICs in these assays can be used to show synergy in the clinical isolate. Shaded blocks represent cell growth and open blocks represent no growth. Assay was performed in triplicate and the data presented is the average of the three assays.

FIG. 21 shows a checkerboard data presentation of inhibition of growth of P. aeruginosa clinical isolate OU19 by a combination of erythromycin and 600-Da BPEI. The MICs in these assays can be used to show synergy in the clinical isolate. Shaded blocks represent cell growth and open blocks represent no growth. Assay was performed in triplicate and the data presented is the average of the three assays.

FIG. 22 shows a checkerboard data presentation of inhibition of growth of E. coli ATCC 25922 by a combination of erythromycin and 600-Da BPEI. The MICs in these assays can be used to show synergy in the clinical isolate. Shaded blocks represent cell growth and open blocks represent no growth. Assay was performed in triplicate and the data presented is the average of the three assays.

FIG. 23 shows a checkerboard data presentation of inhibition of growth of P. aeruginosa clinical isolate OU1 by a combination of azithromycin 600-Da BPEI. The MICs in these assays can be used to show synergy in the clinical isolate. Shaded blocks represent cell growth and open blocks represent no growth. Assay was performed in triplicate and the data presented is the average of the three assays.

FIG. 24 shows a checkerboard data presentation of inhibition of growth of P. aeruginosa clinical isolate OU12 by a combination of azithromycin and 600-Da BPEI. The MICs in these assays can be used to show synergy in the clinical isolate. Shaded blocks represent cell growth and open blocks represent no growth. Assay was performed in triplicate and the data presented is the average of the three assays.

FIG. 25 shows a checkerboard data presentation of inhibition of growth of P. aeruginosa clinical isolate OU15 by a combination of azithromycin and 600-Da BPEI. The MICs in these assays can be used to show synergy in the clinical isolate. Shaded blocks represent cell growth and open blocks represent no growth. Assay was performed in triplicate and the data presented is the average of the three assays.

FIG. 26 shows a checkerboard data presentation of inhibition of growth of P. aeruginosa clinical isolate OU22 by a combination of azithromycin and 600-Da BPEI. The MICs in these assays can be used to show synergy in the clinical isolate. Shaded blocks represent cell growth and open blocks represent no growth. Assay was performed in triplicate and the data presented is the average of the three assays.

FIG. 27 shows a checkerboard data presentation of inhibition of growth of P. aeruginosa ATCC BAA47 by a combination of azithromycin and 600-Da BPEI. The MICs in these assays can be used to show synergy in the clinical isolate. Shaded blocks represent cell growth and open blocks represent no growth. Assay was performed in triplicate and the data presented is the average of the three assays.

### DETAILED DESCRIPTION

The present disclosure is directed to, in at least certain embodiments, compositions which contain branched polyethylenimine (BPEI) compounds (including PEGylated BPEI), and methods of their use for treating inflammation and inflammatory diseases or conditions of epithelia. In certain embodiments, the compositions can be used in treatment of diseases of the skin which involve inflammation (e.g., eczema, psoriasis, dermatitis, and other inflammatory skin diseases), and include ameliorating one or more symptoms of a dermatological pathology associated with skin inflammation in a subject. In certain embodiments, the BPEI compounds can be used as a broad-spectrum anti-inflammatory agent for reducing or inhibiting excessive cytokine release in the skin. For example, the BPEI compounds can suppress or reduce the release of pro-inflammatory cytokines (e.g., IL-8, IL-6, TNF-alpha, and Type-1 interferons) from human epithelial keratinocytes. In one embodiment, the BPEI compound can be used to accelerate wound healing. In other embodiments, the BPEI compounds of the present disclosure can be used to reduce inflammation in lung epithelial tissues such lung tissues comprising bronchial/tracheal epithelial cells and small airway epithelial cells. In one non-limiting embodiment, the BPEI compounds of the present disclosure can be used in treatments for Coronavirus disease-19 (Covid-19). In other embodiments, the BPEI compounds of the present disclosure can be used in treatments for pathogenic airway inflammation, asthma, pulmonary fibrosis, chronic obstructive pulmonary disease, chronic bronchitis, and microbial lung infections including pneumonia. In other embodiments, the BPEI compounds of the present disclosure can be used to reduce inflammation in intestinal epithelial tissues such as small intestine epithelial cells or colon epithelial cells for the treatment of intestinal diseases having an inflammatory component such as ulcerative colitis and inflammatory bowel disease (IBD). In other embodiments, the BPEI compounds of the present disclosure can be used as treatments for septic shock.

In certain embodiments, the present disclosure is directed to potentiated antibiotic compositions for treating strains of carbapenem-resistant Enterobacteriaceae (CRE) species and strains of extended-spectrum β-lactamase (ESBL)-positive bacteria, including, but not limited to, the species Escherichia coli, Klebsiella pneumoniae, Klebsiella oxytoca, Enterobacter aerogenes, Enterobacter cloacae, Citrobacter freundii, Citrobacter koseri, Salmonella enterica, Proteus mirabilis, Serratia marcescens, Morganella morganii, Providencia stuartii, and Providencia rettgeri. The compositions comprise a carbapenem antibiotic and a branched polyethylenimine (BPEI) and/or a polyethylene-BPEI conjugate (a.k.a., PEG-BPEI, or PEGylated-BPEI). Examples of the carbapenem antibiotics that may be used in the composition include, but are not limited to, meropenem, imipenem, ertapenem, doripenem, biapenem, faropenem, tebipenem, and panipenem. In certain embodiments the carbapenem antibiotic and BPEI molecule may be used conjointly with a β-lactamase inhibitor (BLI) such as, but are not limited to, vaborbactam, tazobactam, clavulanic acid (clavulanate), sulbactam, and diazobicyclooctanes such as avibactam and relebactam (MK-7655).

In other non-limiting embodiments, the present disclosure is directed to compositions which contain BPEI compounds in combination with macrolide antibiotics (including but not limited to erythromycin, azithromycin, clarithromycin, fidaxomycin, and roxithromycin) as a treatment to overcome antibiotic resistance mechanisms and potentiate the antibiotic effect against Gram-positive and Gram-negative bacteria, particularly bacteria that demonstrate antimicrobial resistance (AMR). Examples of such bacteria include, but are not limited to, methicillin-resistant Staphylococcus aureus (MRSA) and multidrug-resistant Pseudomonas aeruginosa (MDR-PA) and other listed elsewhere herein. In certain embodiments, the BPEI compound is a BPEI molecule conjugated to a polyethylene glycol (PEG) molecule to form a PEG-BPEI. The BPEI compounds are also effective in suppressing the release of pro-inflammatory cytokines (e.g., IL-8, IL-6, TNF-alpha, and Type-1 interferons, e.g., IFN-α, IFN-β and IFN-γ) from epithelial cells. The compositions can be used to improve and accelerate wound healing by assisting and improving migration of keratinocytes and fibroblasts to and in the wound site, further leading to alleviating and reducing excessive tissue fibrosis and scarring.

Before further describing various embodiments of the compositions, kits and methods of the present disclosure in more detail by way of exemplary description, examples, and results, it is to be understood that the present disclosure is not limited in application to the details of methods and compositions as set forth in the following description. The description provided herein is intended for purposes of illustration only and is not intended to be construed in a limiting sense. The inventive concepts of the present disclosure are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure as defined herein. Thus, while the compositions and methods of the present disclosure have been described in terms of particular embodiments, it will be apparent to those of skill in the art that variations may be applied to the compositions and/or methods and in the steps or in the sequence of steps of the method described herein without departing from the concept, spirit, and scope of the inventive concepts.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference. In particular, incorporated by reference herein in their entireties are PCT Application No. PCT/US2016/037799, filed Jun. 16, 2016, PCT Application No. PCT/US2019/054508, filed Oct. 3, 2019, Published PCT Applications WO/2016/205467A1 and WO2020/081247A1, U.S. patent application Ser. No. 15/736,675, filed Dec. 14, 2017, U.S. patent application Ser. No. 16/530,756, filed Aug. 2, 2019, U.S. patent application Ser. No. 17/208,176 filed Mar. 22, 2021, U.S. patent application Ser. No. 17/231,854, Apr. 15, 2021, U.S. Provisional Application Ser. No. 62/180,976, filed Jun. 17, 2015, U.S. Provisional Application Ser. No. 62/747,517, filed Oct. 18, 2018, each of which may contain subject matter related to the present disclosure.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

Abbreviations

AMR, antimicrobial resistance;

AuPd, Gold palladium;

BPEI, branched polyethylenimine;

BLI, β-lactamase inhibitor;

COVID-19, Coronavirus disease-19;

CAMHB, cation-adjusted Muller-Hinton broth;

CRE, carbapenem-resistant Enterobacteriaceae;

Da, Dalton;

DMSO, dimethylsulfoxide;

EPS, extracellular polymeric substances;

ESBL, extended-spectrum β-lactamase;

FICI, fractional inhibitory concentration index;

HEKa cells, human epithelial keratinocytes cells;

HMDS, hexamethyldisilazane;

IBD, inflammatory bowel disease;

ILs, interleukins;

IL-6, interleukin-6;

IL-1β, interleukin-1β;

IMI, imipenem;

ITC, Isothermal Titration calorimetry;

kDa, kilodaltons;

KPCs, Klebsiella pneumoniae carbapenemases;

LAL, Limulus amebocyte lysate;

LBP, LPS-binding protein;

LPS, lipopolysaccharide;

MBEC, Minimum Biofilm Eradication Concentration;

MBLs, metallo-β-lactamases;

MD-2, myeloid differentiation protein 2;

MDR, multidrug-resistant;

MDR-PA, multidrug-resistant Pseudomonas aeruginosa;

MER, meropenem;

MIC, minimum inhibitory concentration;

MOA, mechanism of action.

MTD, maximum tolerable dose;

MPC₄, Minimum Potentiating Concentration;

MRSA, methicillin-resistant Staphylococcus aureus;

MRSE, methicillin-resistant Staphylococcus epidermidis;

MW, molecular weight;

NDM-1, New-Delhi MBL;

NF-κB, Nuclear factor-κB;

NMR, nuclear magnetic resonance;

NO, Nitric Oxide;

OD₆₀₀, optical density at 600 nm;

ORSA, oxacillin-resistant Staphylococcus aureus;

OXA, oxacillin;

PAMPs, pathogen-associated molecular patterns;

PBP, penicillin-binding protein;

PBS, phosphate-buffered saline;

PC, polycarbonate;

PEG, polyethylene glycol;

PEG-BPEI, polyethylene glycol-branched polyethylenimine;

PG or PGN, peptidoglycan;

PIP, Piperacillin;

PmB, polymyxin-B;

PNAG, poly-N-acetyl glucosamine;

sc, subcutaneous;

SEM, scanning electron microscopy;

SSTI, skin or soft-tissue infections;

TAZO, tazobactam;

TNFs, tumor necrosis factors;

TNFα, tumor necrosis factor alpha;

TLR2, Toll-like receptor 2;

TLR4, Toll-like receptor 4;

TSB, tryptic soy broth;

VRSA, vancomycin-resistant Staphylococcus aureus;

WTA, wall teichoic acid;

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Use of the word “we” as a pronoun herein refers generally to laboratory personnel or other contributors who assisted in laboratory procedures and data collection and is not intended to represent an inventorship role by said laboratory personnel or other contributors in any subject matter disclosed herein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, component, step, feature, structure, or characteristic described in connection with the embodiment is included in at least one embodiment. The appearances of the phrase “in one embodiment” in various places in the specification are not necessarily all referring to the same embodiment.

Use of the word “we” as a pronoun herein refers generally to laboratory personnel or other contributors who assisted in laboratory procedures and data collection and is not intended to represent an inventorship role by said laboratory personnel or other contributors in any particular subject matter disclosed herein.

The term “active agent” as used herein refers to a biologically-active substance, such as, but not limited to, an antibiotic, a BPEI molecule, a PEG-BPEI molecule, a β-lactamase inhibitor, or other biologically active compound or molecule. By “biologically active” is meant the ability of a molecule (the “active agent”) to modify the physiological system of an organism without reference to how the molecule has its physiological effects. The term “BPEI compound” as used herein refers to an active agent which may be either a BPEI molecule or a PEG-BPEI conjugate molecule.

The term “potentiating molecule” refers to an active agent which increases or enhances, i.e., “potentiates,” the effect or effects of a different active agent. For example, as used herein the inhibitory effect or activity of carbapenem antibiotics against CRE bacteria is potentiated by a BPEI compound, or by a BPEI compound in combination with a β-lactamase inhibitor. Likewise, as used herein the term “potentiation” refers to the effect of one or more active agents on a carbapenem antibiotic to increase or enhance the effectiveness or activity of the carbapenem antibiotic. For example, as used herein the inhibitory effect or activity of carbapenem antibiotics against CRE bacteria is potentiated by a BPEI compound, or by a BPEI compound in combination with a β-lactamase inhibitor.

The active agents of the combination therapies of the present disclosure may be used or administered conjointly. As used herein the terms “conjointly” or “conjoint administration” refers to any form of administration of two or more different biologically-active compounds (i.e., active agents) such that the second compound is administered while the previously administered therapeutic compound is still effective in the body, whereby the two or more compounds are simultaneously active in the patient, enabling a synergistic interaction of the compounds. For example, the different therapeutic compounds can be administered either in the same formulation, or in separate formulations, either concomitantly (together) or sequentially. When administered sequentially the different compounds may be administered immediately in succession, or separated by a suitable duration of time, as long as the active agents function together in a synergistic manner. In certain embodiments, the different therapeutic compounds can be administered within one hour of each other, within two hours of each other, within 3 hours of each other, within 6 hours of each other, within 12 hours of each other, within 24 hours of each other, within 36 hours of each other, within 48 hours of each other, within 72 hours of each other, or more. Thus an individual who receives such treatment can benefit from a combined effect of the different therapeutic compounds. In one example of conjoint administration, an antibiotic and a potentiating compound (e.g., a BPEI and/or PEG-BPEI).

The term “pharmaceutically acceptable” refers to compounds and compositions which are suitable for administration to humans and/or animals without undue adverse side effects such as toxicity, irritation and/or allergic response commensurate with a reasonable benefit/risk ratio.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the peptide compound) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The terms “subject” and “patient” are used interchangeably herein and will be understood to refer to a warm-blooded animal, particularly a mammal, and more particularly, humans. Animals which fall within the scope of the term “subject” as used herein include, but are not limited to, dogs, cats, rats, mice, guinea pigs, rabbits, minks, chinchillas, horses, goats, ruminants such as cattle, sheep, swine, poultry such as chickens, geese, ducks, and turkeys, zoo animals, Old and New World monkeys, and non-human primates. Veterinary diseases and conditions which may be treated with the compositions of the present disclosure include, but are not limited to, anthrax, listeriosis, leptospirosis, clostridial and corynebacterial infections, streptococcal mastitis, and keratoconjunctivitis in ruminants; erysipelas, streptococcal and clostridial infections in swine; tetanus, strangles, streptococcal and clostridial infections, and foal pneumonia in horses; urinary tract infections, and streptococcal and clostridial infections in dogs and cats; and necrotic enteritis, ulcerative enteritis and intestinal spirochetosis in poultry.

“Treatment” refers to therapeutic treatments. “Prevention” refers to prophylactic or preventative treatment measures. The term “treating” refers to administering the composition to a subject for a therapeutic purpose or non-therapeutic but beneficial purpose.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent (e.g., a BPEI compound, antibiotic, and/or β-lactamase inhibitor) which is sufficient to exhibit a detectable therapeutic effect, preferably without excessive adverse side effects (such as toxicity, irritation and allergic response) commensurate with a reasonable benefit/risk ratio when used in the manner of the inventive concepts. The therapeutic effect may include, for example but not by way of limitation, a partial or complete elimination of an infection or a partial or complete healing of a wound. The effective amount for a patient will depend upon the type of patient, the patient's size and health, the nature and severity of the condition to be treated, the method of administration, the duration of treatment, the nature of concurrent therapy (if any), the specific formulations employed, and the like. The effective amount for a given situation can be determined by one of ordinary skill in the art using routine experimentation based on the information provided herein. The term “effective amount” refers to an amount of an active agent (e.g., a BPEI compound, with or without an antibiotic) which is sufficient to exhibit a detectable therapeutic effect without excessive adverse side effects (such as toxicity, irritation and allergic response) commensurate with a reasonable benefit/risk ratio when used in the manner of the inventive concepts. The therapeutic effect may include, for example but not by way of limitation, a partial or complete elimination of an infection or wound. The effective amount for a patient will depend upon the type of patient, the patient's size and health, the nature and severity of the condition to be treated, the method of administration, the duration of treatment, the nature of concurrent therapy (if any), the specific formulations employed, and the like. The effective amount for a given situation can be determined by one of ordinary skill in the art using routine experimentation based on the information provided herein.

The term “ameliorate” means a detectable or measurable improvement in a subject's condition, disease, or symptom thereof. A detectable or measurable improvement includes a subjective or objective decrease, reduction, inhibition, closure, suppression, limit, or control in the occurrence, frequency, severity, progression, or duration of the condition or disease, or an improvement in a symptom or an underlying cause or a consequence of the disease, or a reversal of the disease. A successful treatment outcome can lead to a “therapeutic effect” or “benefit” of completely or partially decreasing, reducing, inhibiting, suppressing, limiting, controlling, or preventing the occurrence, frequency, severity, progression, or duration of a disease or condition, or consequences of the disease or condition, such as (but not limited to) a wound or infection, in a subject.

A decrease or reduction in the worsening of a disease or condition, such as stabilizing the condition or disease, such as a wound or infection, is also a successful treatment outcome. A therapeutic benefit therefore need not be complete ablation or reversal of the disease or condition, or of any one of, or most, or all adverse symptoms, complications, consequences or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.), such as partial closure of a wound. Effectiveness of a method or use, such as a treatment that provides a potential therapeutic benefit or improvement of a condition or disease, can be ascertained by various methods, measurements, and testing assays.

The term “wound” as used herein in reference to the skin refers to a break in the continuity of the skin (or other external surface). Wounds are generally further defined as “acute” or “chronic.” An acute wound may be caused by an external force, trauma, or injury. Acute wounds include incisions, excisions (e.g., of extensive skin cancer), tears, punctures, cuts, lacerations, avulsions, necroses, burns from heat or radiation, abrasions, aseptic wounds, contusions, non-penetrating wounds (i.e. wounds in which there is no disruption of the skin but there is injury to underlying structures), open wounds, penetrating wounds, perforating wounds, puncture wounds, septic wounds, sores, subcutaneous wounds, and can be caused accidentally or purposefully (e.g., by surgery), deep fungal and bacterial infections, vasculitis, scleroderma, pemphigus, and toxic epidermal necrolysis. Examples of sores include pressure sores (e.g., bed sores), cancer sores, chrome sores, and cold sores. The wound may be located internally or externally of a subject. A chronic wound is generally defined as a wound which is unhealed after at least three weeks to three months, or a wound that does not completely heal, or that has failed to proceed through an orderly and timely process to produce sustained anatomic and functional integrity through the repair process. A chronic wound may be derived from an acute wound which has become infected causing the wound to become inflamed thereby interrupting the normal healing process. In certain cases, chronic wounds are referred to herein as ulcers. In general, an ulcer is defined as a breakdown or gradual disturbance in the skin (or other external surface) tissue caused by an underlying (internal) etiology or pathology.

The wound may also be due to a disorder (such as diabetes), an infectious lesion, surgery, or a puncture. The wound may be due to destructive wound inflammation, delayed or impaired healing, or disturbed tissue regeneration. The term “accelerating wound healing” or “acceleration of wound healing” refers to the increase in the rate of healing, e.g., a reduction in time until complete wound closure occurs or a reduction in time until a % reduction in wound area occurs.

The term “ulcer” refers a site of damage to the skin or mucous membrane that is often characterized by the formation of pus, death of tissue (necrosis), and is accompanied by an inflammatory reaction. Ulcers are frequently chronic wounds. A “diabetic wound” is a wound that is associated with diabetes. A “diabetic ulcer” is an ulcer that is associated with diabetes. A “chronic wound” refers a wound that has not healed within three months. Chronic wounds include, but are not limited to, e.g., arterial ulcers, diabetic ulcers, pressure ulcers or bed sores, and venous ulcers. An acute wound can develop into a chronic wound. A “dermal wound” refers to a lesion in one or more layers of skin of a subject, e.g., wherein the lesion comprises one or more apoptotic dermal cells and/or one or more necrotic dermal cells. The term “dermal wound” shall be taken to include a wound that affects an epidermal layer of a subject and/or a dermal layer of a subject and/or a hypodermal layer of a subject. The term “healing” in the context of the present disclosure is a promotion or acceleration of the time from when the compound is administered until significant or complete wound closure (full wound contraction). A non-diabetic wound is a wound that occurs in a subject who does not have diabetes.

Wounds may be caused by, or may become chronic due to, ischemia. Ischemia is caused by limited blood supply to a wound site causing a shortage of oxygen and other necessary blood-borne products required by the tissue due to increased metabolic costs of healing. Peripheral vascular disease or disruption is a common cause of ischemic wounds. Individuals with poor peripheral circulation are at high risk for developing ischemic chronic wounds. Other medical conditions associated with ischemic wounds include diabetes mellitus, renal failure, hypertension, lymphedema, inflammatory diseases such as vasculitis or lupus, and current or past tobacco use. Ischemic wounds thus refer to wounds to which the flow of blood has been obstructed, restricted, or otherwise impaired, such that the wound site is deprived of oxygen and nutrients. Damaged tissue deprived of adequate blood flow has a decreased ability to heal, and as such predisposes individuals to the development of chronic wounds.

In certain embodiments, the compounds and compositions disclosed herein may be used to treat and promote healing of wounds in subjects who are otherwise healthy, i.e., subjects who do not have chronic conditions which impair wound healing (such as, but not limited to, diabetes). Examples of such wounds in otherwise healthy subjects include, but are not limited to, surface wounds such as lacerations, abrasions, avulsions, incisions, and amputations, and other wounds described above or elsewhere herein. In certain embodiments, the compounds and compositions are used to enhance and/or promote healing of acute wounds of a non-diabetic nature (e.g., lacerations, abrasions, avulsions, incisions, amputations, and burns).

In certain embodiments, the compounds and compositions disclosed herein may be used to treat and promote healing of chronic or non-healing wounds such as (but not limited to) diabetic wounds and ulcers (for example of the legs and feet) in subjects having diabetes, or wounds due to peripheral vascular disease or cardiovascular disease in subjects who have chronic conditions which impair wound healing. Thus, in certain embodiments, the compounds and compositions of the present disclosure are used to enhance and/or promote healing of chronic wounds.

As noted elsewhere herein, a successful treatment does not require complete healing or closure of a wound but may comprise an amelioration of the wound, for example a partial closure of the wound or partial epithelialization of the wound. The compounds and compositions in certain embodiments may be used as treatments to enhance acceptance of grafts such as skin grafts.

The compounds and compositions may be used in combination with each other, in combination with other antibiotics, or in combination with another drug given to treat a particular condition.

In certain embodiments, active agents such as the BPEI compounds of the present disclosure can be used to treat inflammatory skin diseases or conditions. Where used herein, the term inflammatory skin disease or condition is defined as a skin disease or condition characterized by the production of pro-inflammatory cytokines. Examples of such diseases and conditions include, but are not limited to, eczema (atopic dermatitis), seborrheic dermatitis, psoriasis, urticaria (hives), rosacea, acne, allergic contact dermatitis, irritant contact dermatitis, pruritus, xerosis, pyoderma gangrenosum, lichenoid disorders such as lichen planus, bullous diseases such as bullous pemphigoid, cutaneous vasculitis, granuloma annulare, keloid formation, abnormalities in skin pigmentation, actinic keratosis, actinic elastosis, and granulomatous skin diseases. In certain embodiments, the psoriasis comprises plaque psoriasis, guttate psoriasis, inverse psoriasis, pustular psoriasis, or erythrodermic psoriasis, or any combination thereof. The psoriasis may be mild, moderate or severe. In at least certain embodiments, the active agents are contained in a composition with a carrier or vehicle described elsewhere herein for topical administration.

In certain embodiments, the method of the present disclosure is directed to treating, inhibiting, preventing, reducing the severity of, reducing the incidence of, delaying the onset of, reducing pathogenesis, and/or reducing at least one symptom of the inflammatory skin disease or condition. In certain embodiments, the at least one symptom of the inflammatory skin disease or condition or lesion comprises scaly patches on the skin, itchy skin, burning skin, stinging skin or pain, abnormal scar formation, abnormal pigmentation, skin creases, sun exposure damage, or any combination thereof. In certain embodiments, the inflammatory skin disease or condition is present at least on an outside surface of skin. In certain embodiments, a skin surface comprises an elbow, knee, hand, finger, leg, foot, face, nail, genital, or scalp, or any combination thereof. In certain embodiments, wherein the inflammatory skin disease o condition comprises a lesion, the methods of the present disclosure is directed to treating, reducing the severity of, reducing the incidence of, delaying the onset of, or reducing pathogenesis of the lesion (s), or reducing the number of lesions, the size of the lesions, the spread of a lesion, increasing the healing of a lesion, or reducing the depth of the lesion, or any combination thereof.

In certain embodiments of the present disclosure, the BPEI compounds may be used in association with an antibiotic, such as a β-lactam antibiotic. The term “β-lactam antibiotic” refers to the class of antibiotic agents that have a β-lactam ring or derivatized β-lactam ring in their molecular structures. Examples of such β-lactam antibiotics include but are not limited to, penams, including but not limited to, penicillin, benzathine penicillin, penicillin G, penicillin V, procaine penicillin, ampicillin, amoxicillin, Augmentin® (amoxicillin+clavulanic acid), methicillin, cloxacillin, dicloxacillin, flucloxacillin, nafcillin, oxacillin, temocillin, mecillinam, carbenicillin, ticarcillin, and azlocillin, mezlocillin, piperacillin, Zosyn® (piperacillin+tazobactam); cephems, including but not limited to, cephalosporin C, cefoxitin, cephalosporin, cephamycin, cephem, cefazolin, cephalexin, cephalothin, cefaclor, cefamandole, cefuroxime, cefotetan, cefoxitin, cefixime, cefotaxime, cefpodoxime, ceftazidime, ceftriaxone, cefepime, cefpirome, and ceftaroline; carbapenems and penems including but not limited to, biapenem, doripenem, ertapenem, earopenem, imipenem, primaxin, meropenem, panipenem, razupenem, tebipenem, and thienamycin; and monobactams including but not limited to, aztreonam, tigemonam, nocardicin A, and tabtoxinine β-lactam.

The terms “effective amount”, “antibacterially-effective amount”, or “therapeutically-effective amount” refers to an amount of an antibiotic composition (e.g., an antibiotic plus BPEI compound) which is sufficient to exhibit a detectable therapeutic effect against bacterial growth and/or inflammation without excessive adverse side effects (such as toxicity, irritation and allergic response) commensurate with a reasonable benefit/risk ratio when used in the manner as described herein. The effective amount for a patient will depend upon the type of patient, the patient's size and health, the nature and severity of the condition to be treated, the method of administration, the duration of treatment, the nature of concurrent therapy (if any), the specific formulations employed, and the like. The effective amount for a given situation can be determined by one of ordinary skill in the art using routine experimentation based on the information provided herein.

The term BPEI refers to a branched poly(ethylenimine), for example as further described below as to molecular weight (Mw). As noted above the term BPEI or BPEI compound as used herein is also intended to include reference to a PEGylated BPEI molecule (PEG-BPEI), i.e., a PEG molecule conjugated to a BPEI molecule, unless specifically indicated otherwise. Polyethylene glycol (PEG) molecules are described in further detail below.

In some embodiments of the present disclosure, a low molecular weight (“low Mw”) B PEI is used in combination with an anti-bacterial agent to treat and/or inhibit a resistant bacterial infection and/or the growth of resistant bacterial infection, e.g., by sensitizing a bacterium that was previously resistant or substantially resistant to an antibacterial agent, are described herein. In certain non-limiting embodiments the low Mw BPEI of the present disclosure has a Mw in range of, for example, 0.1 kDa (kilodaltons) to 25 kDa, e.g., 500-1000 Da. Examples of BPEI compounds which may be used in various embodiments of the present disclosure include but are not limited to those shown in U.S. Pat. Nos. 7,238,451 and 9,238,716, and U.S. Published application 2014/0369953, the entireties of which are hereby incorporated by reference herein.

A minimum inhibitory concentration (MIC) of an antibiotic for a particular bacterial strain is defined as the lowest concentration of the antibiotic that is required to inhibit the growth of the bacterium. The MIC is determined by finding the concentration of antibiotic at which there is no growth of the bacterium.

A breakpoint (or resistance breakpoint) is defined as a concentration (mg/L) of an antibiotic that defines when a strain of bacteria is susceptible to successful treatment by the antibiotic. If the MIC is less than or equal to the breakpoint, the strain is considered susceptible to the antibiotic. If the MIC is greater than the breakpoint, the strain is considered intermediate or resistant to the antibiotic.

Sensitizing, or sensitization, as the term is used herein, is the process of lowering the MIC of an antibiotic for a resistant bacterial strain to a value that is below the resistance breakpoint for the bacterial strain, thereby causing the bacterium to be more susceptible to that antibiotic.

The compounds and compositions of the present disclosure can be used to treat a subject having resistant bacterial infection, e.g., by administering a BPEI in combination with an antibiotic. The combinations of the BPEI and the antibacterial agent can result in sensitization of a resistant bacterial strain, e.g., the resistant bacterial strain has a reduced MIC of either the BPEI, or the antibacterial agent, or both, so that the MIC is below the resistance breakpoint for the bacterial strain. As noted above, a beta lactamase inhibitor may be used conjointly with the BPEI compound/antibiotic combination.

As used herein or “resistance” or “resistant” refers to the lack of killing or inhibitory effectiveness of a particular antibacterial agent against a particular bacterial strain. As used herein “resistant bacterial strain” means a bacterial strain which is resistant to an antibacterial agent, e.g. having an MIC that is greater than the resistance breakpoint (as the term is defined herein). In certain embodiments the MIC of a resistant bacterial strain will be at least 2-fold, 4-fold, 8-fold, 10-fold, 16-fold, 32-fold, 64-fold, or 100-fold greater than for that seen with a non-resistant bacterial strain for a selected antibacterial agent. As used herein, rendering or transforming a resistant bacterial into a sensitive bacterial strain means reducing the MIC, e.g., by at least 2-fold, 4-fold, 8-fold, 10-fold, 16-fold, 32-fold, 64-fold, or 100-fold.

The term “biofilm” as used herein refers to an aggregate of microorganisms in which cells adhere to each other and/or to a surface. These adherent cells are frequently embedded within a self-produced matrix of extracellular polymeric substance. The microorganisms comprising a biofilm may include bacteria, archaea, fungi, protozoa, algae, or combinations thereof. In particular embodiments, the biofilm comprises a bacterium (such as described elsewhere herein) such that the biofilm is a bacterial biofilm.

In some embodiments of the present disclosure, the surface having the biofilm thereon is a surface of or within the body of a subject. For example, the surface may be an epithelial surface of an external wound.

In some embodiments, the surface having the biofilm thereon may be a surface of a medical device. In some embodiments, the biofilm may be partially or entirely implantable in a body of a subject. For example, the medical device may be a catheter. Non-limiting examples of suitable catheters include intravascular catheters (such as, e.g., arterial catheters, central venous catheters, hemodialysis catheters, peripheral and venous catheters), endovascular catheter microcoils, peritoneal dialysis catheters, urethral catheters, catheter access ports, shunts, intubating and tracheotomy tubes. For example, the medical device may be a peripherally inserted central catheter (PICC) line. In another embodiment, the implantable device may be a cardiac device. Examples of cardiac devices include, but are not limited to, cardiac stents, defibrillators, heart valves, heart ventricular assist devices, OEM component devices, pacemakers, and pacemaker wire leads. In further embodiments, the medical device may be an orthopedic device. Non-limiting examples of suitable orthopedic devices include implants such as knee replacements, hip replacements, shoulder replacements, other joint replacements and prostheses, spinal disc replacements, orthopedic pins, plates, screws, rods, and orthopedic OEM components. In other embodiments, the medical device may include endotracheal tubes, nasogastric feeding tubes, gastric feeding tubes, synthetic bone grafts, bone cement, biosynthetic substitute skin, vascular grafts, surgical hernia mesh, embolic filter, ureter renal biliary stents, urethral slings, gastric bypass balloons, gastric pacemakers, insulin pumps, neurostimulators, penile implants, soft tissue silicone implants, intrauterine contraceptive devices, cochlear implants, dental implants and prosthetics, voice restoration devices, ophthalmic devices such as contact lenses.

The subject having the surface may be a veterinary subject. Non-limiting examples of suitable veterinary subjects include companion animals such as cats, dogs, rabbits, horses, and rodents such as gerbils; agricultural animals such as cows, cattle, pigs, goats, sheep, horses, deer, chickens and other fowl; zoo animals such as primates, elephants, zebras, large cats, bears, and the like; and research animals such as rabbits, sheep, pigs, dogs, primates, chinchillas, guinea pigs, mice, rats and other rodents. For instance, the composition may be used to treat skin infections, soft tissue infections, and/or mastitis in veterinary subjects such as companion animals and/or agricultural animals. The veterinary subject may be suffering from or diagnosed with a condition needing treatment, or the veterinary subject may be treated prophylactically.

In other embodiments, the subject having the surface having the biofilm thereon may be a surface on or within a human health care patient. Non-limiting examples of suitable health care patients include ambulatory patients, surgery patients, medical implantation patients, hospitalized patients, long-term care patients, and nursing home patients. In still other embodiments, the subject may be a health care worker. Suitable health care workers include those with direct and indirect access to patients, medical equipment, and medical facilities. The surface may be a surface of a wound such as described elsewhere herein.

In some embodiments, the combination of the BPEI and the antibiotic results in a reduction in the MIC of the BPEI and/or the antibiotic of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100.5%, or more.

The antimicrobial (antibacterial) action of two or more active agents is considered additive if the combined action merely results from the addition of the effects the individual components would have in isolation. In contrast, the antimicrobial action of two or more active compounds is considered to be synergistic if the combined effect of the two or more compounds is stronger than expected based on the assumption of additivity, i.e., when the combined action of the two or more compounds results in an effect greater that the the additive sum of the individual effects.

The terms “synergy” or “synergistic,” as in “synergistic effect” or “synergistic activity,” refers to an effect in which two or more agents work together to produce an effect that is more than additive of the effects of each agent, independently. More particularly, the terms “synergy”, “synergistic”, “synergistic effect” or “synergistic activity” as used herein, refers to an outcome when two agents (e.g., BPEI and an antibiotic) are used in combination, wherein the combination of the agents acts so as to require a smaller amount of each individual agent than would be required of that agent to be efficacious in the absence of the other agent. For example, with lower dosages of the first agent than would be required in the absence of the second agent. In some embodiments, use of synergistic agents can result in the beneficial effect of less overall use of an agent. Typically, evidence of synergistic antimicrobial action may be provided at concentrations below the MICs of each of the components when taken individually. However, a synergistic interaction can also occur when the concentration of one or more of the active compounds is raised above its MIC (when taken individually).

The fractional inhibitory concentration (FIC) as used herein is a measure of the interaction of two agents, such as an antibiotic and a BPEI compound, used together, and is an indicator of synergy. FIC uses a value of the MIC of each of the independent agents, e.g., MIC_{A} and MIC_{B} for agents A and B, for a particular bacterium as the basis, then takes the concentration of each component in a mixture where an MIC_{(A in B)} is observed. For example, for a two component system of agents A and B, MIC_{(A in B)} is the concentration of A in the compound mixture and MIC_{(B in A)} is the concentration of B in the compound mixture. The FIC is defined as follows:

FIC_{A}=(MIC_{(A in B)}/MIC_{A}) Eqn. 1

FIC_{B}=(MIC_{(B in A)}/MIC_{B}) Eqn. 2

FIC_{A+B}=FIC_{A}+FIC_{B} Eqn. 3

Synergism (i.e., the two compounds together provide a synergistic effect or synergistic activity against a bacterium) is defined herein as occurring when FIC_{A+B}≤0.5. The mixture is defined as having an additive effect when 1≤FIC_{A+B}≤4. When, FIC_{A+B}>4 the mixture is considered to have an antagonistic interaction. An example of how FIC is used to determine synergism is shown in U.S. Pat. No. 8,338,476, the entirety of which is incorporated herein by reference in its entirety.

In certain embodiments of the present disclosure, the BPEI compound/antibiotic combination results in an FIC less than about 0.55, or less than about 0.5, or less than about 0.4, or less than about 0.3, or less than about 0.2, or less than about 0.1, or less than about 0.05, or less than about 0.02, or less than about 0.01, or less than about 0.005, or less than about 0.001. In some embodiments, the combination results in a bactericidal activity at least about 2 logs, at least about 2.5 logs, at least about 3 logs, at least about 3.5 logs, at least about 4 logs, at least about 4.5 logs, or at least about 5 logs more effective than the most effective individual activity, e.g., the activity of the BPEI compound or the antibiotic agent.

As used herein, “resistant microorganism or bacterium” means an organism which has become resistant to an anti-bacterial agent. In certain embodiments an MIC of a resistant bacterium will be at least, 2-fold, 4-fold, 8-fold, 10-fold, 16-fold, 32-fold, 64-fold, or 100-fold greater than that seen with a non-resistant bacterium for a particular anti-bacterial agent. As used herein, the term “resistance breakpoint” is the threshold concentration of an antibacterial agent above which a bacterium is considered resistant, as defined above.

In certain non-limiting embodiments, the antibiotic/BPEI composition is formulated to contain a mass ratio in a range of 100:1 (e.g., 100 mg antibiotic per 1 mg of BPEI), to 1:1 (1 mg antibiotic per 1 mg BPEI), to 1:100 (1 mg antibiotic per 100 mg BPEI), or more particularly, a mass ratio in a range of 75:1 (e.g., 75 mg antibiotic per 1 mg of BPEI), to 1:1 (1 mg antibiotic per 1 mg BPEI), to 1:75 (1 mg antibiotic per 75 mg BPEI), or more particularly, a mass ratio in a range of 64:1 (e.g., 64 mg antibiotic per 1 mg of BPEI), to 1:1 (1 mg antibiotic per 1 mg BPEI), to 1:64 (1 mg antibiotic per 64 mg BPEI), or more particularly, a mass ratio in a range of 50:1 (e.g., 50 mg antibiotic per 1 mg of BPEI), to 1:1 (1 mg antibiotic per 1 mg BPEI), to 1:50 (1 mg antibiotic per 50 mg BPEI), or more particularly, a mass ratio in a range of 32:1 (e.g., 32 mg antibiotic per 1 mg of BPEI), to 1:1 (1 mg antibiotic per 1 mg BPEI), to 1:32 (1 mg antibiotic per 32 mg BPEI), or more particularly, a mass ratio in a range of 24:1 (e.g., 24 mg antibiotic per 1 mg of BPEI), to 1:1 (1 mg antibiotic per 1 mg BPEI), to 1:24 (1 mg antibiotic per 24 mg BPEI), or more particularly, a mass ratio in a range of 16:1 (e.g., 16 mg antibiotic per 1 mg of BPEI), to 1:1 (1 mg antibiotic per 1 mg BPEI), to 1:16 (1 mg antibiotic per 16 mg BPEI), or more particularly, a mass ratio in a range of 10:1 (e.g., 10 mg antibiotic per 1 mg of BPEI), to 1:1 (1 mg antibiotic per 1 mg BPEI), to 1:10 (1 mg antibiotic per 10 mg BPEI), or more particularly, a mass ratio in a range of 8:1 (e.g., 8 mg antibiotic per 1 mg of BPEI), to 1:1 (1 mg antibiotic per 1 mg BPEI), to 1:8 (1 mg antibiotic per 8 mg BPEI), or more particularly, a mass ratio in a range of 4:1 (e.g., 4 mg antibiotic per 1 mg of BPEI), to 1:1 (1 mg antibiotic per 1 mg BPEI), to 1:4 (1 mg antibiotic per 4 mg BPEI), or any range comprising a combination of said ratio endpoints, such as for example, a mass ratio in a range of 64:1 (e.g., 64 mg antibiotic per 1 mg of BPEI), to 1:4 (1 mg antibiotic per 4 mg BPEI), or a mass ratio in a range of 32:1 (e.g., 32 mg antibiotic per 1 mg of BPEI), to 1:16 (1 mg antibiotic per 16 mg BPEI). As noted above, the term BPEI may also refer to a PEG-BPEI.

In certain non-limiting embodiments, the dosage of the BPEI compound or antibiotic/BPEI composition administered to a subject could be in a range of 1 μg per kg of subject body mass to 1000 mg/kg, or in a range of 5 μg per kg to 500 mg/kg, or in a range of 10 μg per kg to 300 mg/kg, or in a range of 25 μg per kg to 250 mg/kg, or in a range of 50 μg per kg to 250 mg/kg, or in a range of 75 μg per kg to 250 mg/kg, or in a range of 100 μg per kg to 250 mg/kg, or in a range of 200 μg per kg to 250 mg/kg, or in a range of 300 μg per kg to 250 mg/kg, or in a range of 400 μg per kg to 250 mg/kg, or in a range of 500 μg per kg to 250 mg/kg, or in a range of 600 μg per kg to 250 mg/kg, or in a range of 700 μg per kg to 250 mg/kg, or in a range of 800 μg per kg to 250 mg/kg, or in a range of 900 μg per kg to 250 mg/kg, or in a range of 1 mg per kg to 200 mg/kg, or in a range of 1 mg per kg to 150 mg/kg, or in a range of 2 mg per kg to 100 mg/kg, or in a range of 5 mg per kg to 100 mg/kg, or in a range of 10 mg compound per kg to 100 mg/kg, or in a range of 25 mg per kg to 75 mg/kg. For example, in certain non-limiting embodiments, the composition could contain antibiotic in a range of 0.1 mg/kg to 10 mg/kg, and BPEI in a range of 0.1 mg/kg to 10 mg/kg, or any range comprising a combination of said ratio endpoints, such as, for example, a range of 10 μg/kg to 10 mg/kg of the antibiotic/BPEI composition. In some embodiments, the antibiotic and/or potentiating compound is administered at a dose of about 0.1 mg/kg to about 50 mg/kg. In particular embodiments, the subject is a pediatric patient, which means under 18 years of age for a human patient. For a pediatric patient, in some embodiments the antibiotic and/or potentiating compound is administered about 10 mg/kg to about 50 mg/kg intravenously or intramuscularly every 6 to 12 hours or about 12.5 mg/kg orally every 6 hours.

The BPEI portion of the BPEI compounds used in the present formulations (e.g., the BPEI portion of a PEG-BPEI) may have an average molecular weight (MW) in a range of, for example, from 0.1 kDa (kilodaltons), to 0.2 kDa, to 0.3 kDa, to 0.4 kDa, to 0.50 kDa, to 0.6 kDa, to 0.7 kDa, to 0.8 kDa, to 0.9 kDa, to 1.0 kDa, to 1.1 kDa, to 1.2 kDa, to 1.3 kDa, to 1.4 kDa, to 1.5 kDa, to 1.6 kDa, to 1.7 kDa, to 1.8 kDa, to 1.9 kDa, to 2 kDa, to 2.5 kDa, to 3 kDa, to 3.5 kDa, to 4 kDa, to 4.5 kDa, to 5 kDa, to 5.5 kDa, to 6 kDa, to 6.5 kDa, to 7 kDa, to 7.5 kDa, to 8 kDa, to 9 kDa, to 10 kDa, to 12.5 kDa, to 15 kDa, to 17.5 kDa, to 20 kDa, to 22.5 kDa, to 25 kDa, to 30 kDa, to 35 kDa, to 40 kDa, to 45 kDa, to 50 kDa, to 55 kDa, to 60 kDa, to 65 kDa, to 70 kDa, to 75 kDa including any fractional or integeric value within said range. Also, the percentage of primary amine-to-secondary amine-to-tertiary amine in the BPEI can be varied. For example, the BPEI may have a higher primary amine content as compared to the secondary amine and/or tertiary amine content.

The PEG molecules used as the PEG portion of a PEG-BPEI in the present formulations (or as carriers) may have an average molecular weight (MW) in a range of, for example, from 0.1 kDa (kilodaltons), to 0.2 kDa, to 0.3 kDa, to 0.4 kDa, to 0.50 kDa, to 0.6 kDa, to 0.7 kDa, to 0.8 kDa, to 0.9 kDa, to 1.0 kDa, to 1.1 kDa, to 1.2 kDa, to 1.3 kDa, to 1.4 kDa, to 1.5 kDa, to 1.6 kDa, to 1.7 kDa, to 1.8 kDa, to 1.9 kDa, to 2 kDa, to 2.1 kDa, to 2.2 kDa, to 2.3 kDa, to 2.4 kDa, to 2.5 kDa, to 2.6 kDa, to 2.7 kDa, to 2.8 kDa, to 2.9 kDa, to 3 kDa, to 3.1 kDa, 3.2 kDa, to 3.3 kDa, to 3.4 kDa, to 3.5 kDa, to 3.6 kDa, to 3.7 kDa, to 3.8 kDa, to 3.9 kDa, to 4 kDa, to 4.1 kDa, 4.2 kDa, to 4.3 kDa, to 4.4 kDa, to 4.50 kDa, to 4.6 kDa, to 4.7 kDa, to 4.8 kDa, to 4.9 kDa, to 5 kDa, to 5.5 kDa, to 6 kDa, to 6.5 kDa, to 7 kDa, to 7.5 kDa, to 8 kDa, to 9 kDa, to 10 kDa, including any fractional or integeric value within said range, such as 150 Da to 2500 Da (i.e., 0.15 kDa to 2.5 kDa), 200 Da to 1750 Da (i.e., 0.2 kDa to 1.75 kDa), 250 Da to 1500 Da (i.e., 0.25 kDa to 1.5 kDa), and 300 Da to 1250 Da (i.e., 0.3 kDa to 1.25 kDa).

The BPEI compounds of the present disclosure can be delivered as a dosage in a formulation without an additional active agent, or can be delivered as a dosage with an additional active agent, such as an antibiotic. In embodiments in which an antibiotic and BPEI compound are used together, the antibiotic and BPEI compound can be administered together in a single formulation (dose), or together (simultaneously) in separate formulations (doses), or sequentially, whereby administration of the antibiotic dosage is followed by the BPEI dosage, or administration of the BPEI dosage is followed by administration of the antibiotic dosage.

The BPEI compound (alone, or used conjointly with an antibiotic, or other active agent) can be administered, for example but not by way of limitation, on a one-time basis, or administered at multiple times (for example but not by way of limitation, from one to five times per day, or once or twice per week), or continuously via a venous drip, depending on the desired therapeutic effect. In one non-limiting example of a therapeutic method of the present disclosure, the composition is provided in an IV infusion. Administration of the compounds used in the pharmaceutical composition or to practice the method of the present disclosure can be carried out in a variety of conventional ways, such as, but not limited to, topically, orally, by inhalation, rectally, or by cutaneous, subcutaneous, intraperitoneal, vaginal, or intravenous injection. Oral formulations may be formulated such that the compounds pass through a portion of the digestive system before being released, for example it may not be released until reaching the small intestine, or the colon. When the BPEI formulations (with or without an antibiotic) of the present disclosure are delivered by inhalation, they may be delivered via a soft mist nebulizer (e.g., a jet, ultrasonic, or vibrating-mesh nebulizer), a pressurized metered-dose inhaler (MDI), or a dry powder inhaler (DPI), or by any other suitable means, e.g., via a catheter inserted directly into the lung, or via a ventilator when a patient himself or herself is unable to inhale voluntarily.

In some embodiments, when the BPEI compound is used in conjunction with an antibiotic, the BPEI compound and the antibiotic are provided in the same composition. In other embodiments the antibiotic and the BPEI compound are administered simultaneously, or nearly simultaneously, but in different compositions or dosage forms. A subject is administered an antibiotic up to 24 hours prior to administration of the BPEI compound in some cases. In others, the BPEI compound is administered up to 24 hours prior to administration of the antibiotic. In some embodiments, the antibiotic and BPEI compound are administered within 0.5, 1, 2, 3, 4, 6, 8, 10, 12, 14, 16, 18, 20, 22, or 24 hours of each other.

As noted above, in certain embodiments, the compositions of the present disclosures may be applied topically to an external or internal wound to treat a planktonic or biofilm bacterial infection in or on the wound and/or to reduce inflammation in the wound. The treated wounds may be acute wounds, such as abrasions, lacerations, punctures, avulsions and incisions, or other acute wounds as described herein, or may be chronic wounds, such as non-diabetic chronic wounds, or diabetic chronic wounds including but not limited to diabetic foot and leg ulcers, venous leg ulcers, pressure ulcers (e.g., bed sores), wounds due to arterial insufficiency, wounds caused by burns or radiation, and chronic surgical wounds (e.g., due to abdominal surgery), or other chronic wounds as described herein. The BPEI compound may be supplied without an antibiotic, or with an antibiotic. When used with an antibiotic, the BPEI compound may be supplied separately, or with the antibiotic, as explained elsewhere herein.

In certain embodiments, the methods of the present disclosure include topical, transdermal, sub-dermal, enteral, parental or intravenous administration of BPEI-containing compositions. For example, topical administration of the BPEI compound may comprise the administration of a cream, gel, ointment, spray, lip-balm, balm, emulsion, liposome, liquid crystal preparation or lotion, or any combination thereof. In one embodiment, administration comprises an at least once a day administration for one or more days (e.g., 1-30 days) until at least one symptom of the inflammatory disease or condition is alleviated. In another embodiment, administration comprises an at least twice a day administration for one or more days (e.g., 1-30 days) until at least one symptom of the inflammatory disease or condition is alleviated. In another embodiment, administration comprises an at least 3-6 times per day administration for one or more days (e.g., 1-30 days) until at least one symptom of the inflammatory disease or condition is alleviated.

The composition for topical or internal application may be provided in any suitable solid, semi-solid, or liquid form. In certain embodiments, the topical composition may be provided in or be disposed in a carrier(s) or vehicle(s) such as, for example, creams, pastes, gums, lotions, gels, foams, ointments, emulsions, suspensions, aqueous solutions, powders, lyophilized powders, solutions, granules, foams, drops, eye drops, adhesives, sutures, aerosols, sprays, sticks, soaps, bars of soap, balms, body washes, rinses, tinctures, gel beads, gauzes, wound dressings, bandages, cloths, towelettes, stents, and sponges. Non-limiting examples of formulations of such carriers and vehicles include, but are not limited to, those shown in “Remington, The Science and Practice of Pharmacy, 22nd ed., 2012, edited by Loyd V. Allen, Jr”.

Creams are emulsions of water in oil (w/o), or oil in water (o/w). O/w creams spread easily and do not leave the skin greasy and sticky. W/o creams tend to be more greasy and more emollient. Ointments are semi-solid preparations of hydrocarbons and the strong emollient effect makes it useful in cases of dry skin. The occlusive effect enhances penetration of the active agent and improves efficacy. Pastes are mixtures of powder and ointment. The addition of the powder improves porosity thus breathability. The addition of the powder to the ointment also increases consistency so the preparation is more difficult to rub off or contact non-affected areas of the skin. Lotions are liquid preparations in which inert or active medications are suspended or dissolved. For example, an o/w emulsion with a high water content gives the preparation a liquid consistency of a lotion. Most lotions are aqueous of hydroalcoholic systems wherein small amounts of alcohol are added to aid in solubilization of the active agent and to hasten evaporation of the solvent from the skin surface. Gels are transparent preparations containing cellulose ethers or carbomer in water, or a water-alcohol mixture. Gels liquefy on contact with the skin, dry, and leave a thin film of active medication.

A person with ordinary skill in the art will be capable of determining the effective amount of the composition needed for a particular treatment. Such amount may depend on the strength of the composition or extent of the wound to be treated. Although a person with ordinary skill in the art will know how to select a treatment regimen for a specific condition. In a non-limiting example, a dosage of the composition comprising about 0.01 mg to about 1000 mg of the active agent (antibiotic plus BPEI) per ml may be applied 1 to 2 to 3 to 4 to 5 to 6 times per day or more to the affected area. It is foreseeable in some embodiments that the composition is administered over a period of time. The composition may be applied for a day, multiple days, a week, multiple weeks, a month, or even multiple months in certain circumstances. Alternatively, the composition may be applied only once when the skin condition is mild.

In certain embodiments, the composition may comprise the active agents in a concentration of, but is not limited to, 0.0001 M to 1 M, for example, or 0.001 M to 0.1 M. The composition may comprise about 0.01 to about 1000 milligrams of the active agents per ml of carrier or vehicle with which the active agents are combined in a composition or mixture. The composition may comprise about 1 wt % to about 90 wt % (or 1 mass % to about 90 mass %) of one or more shikimate analogues and about 10 wt % to about 99 wt % (or 10 mass % to about 99 mass %) of one or more secondary compounds (where “wt %” is defined as the percentage by weight of a particular compound in a solid or liquid composition, and “mass %” is defined as the percentage by mass of a particular compound in a solid or liquid composition).

The topical compositions may further comprise ingredients such as propylene glycol, sodium stearate, glycerin, a surfactant (e.g., sodium laurate, sodium laureth sulfate, and/or sodium lauryl sulfate), and water, and optionally, sorbitol, sodium chloride, stearic acid, lauric acid, aloe vera leaf extract, pentasodium penetrate, and/or tetrasodium etidronate.

The topical compositions may be formulated with liquid or solid emollients, solvents, thickeners, or humectants. Emollients include, but are not limited to, stearyl alcohol, mink oil, cetyl alcohol, oleyl alcohol, isopropyl laurate, polyethylene glycol, olive oil, petroleum jelly, palmitic acid, oleic acid, and myristyl myristate. Emollients may also include natural butters extracted from various plants, trees, roots, or seeds. Examples of such butters include, but are not limited to, shea butter, cocoa butter, avocado butter, aloe butter, coffee butter, mango butter, or combination thereof.

Suitable materials which may be used in the compositions as carriers or vehicles or secondary compounds or solvents include, but are not limited to, propylene glycol, ethyl alcohol, isopropanol, acetone, diethylene glycol, ethylene glycol, dimethyl sulfoxide, and dimethyl formamide. Suitable humectants include, but are not limited to, acetyl arginine, algae extract, Aloe barbadensis leaf extract, 2,3-butanediol, chitosan lauroyl glycinate, diglycereth-7 malate, diglycerin, diglycol guanidine succinate, erythritol, fructose, glucose, glycerin, honey, hydrolyzed wheat protein/polyethylene glycol-20 acetate copolymer, hydroxypropyltrimonium hyaluronate, inositol, lactitol, maltitol, maltose, mannitol, mannose, methoxypolyethylene glycol, myristamidobutyl guanidine acetate, polyglyceryl sorbitol, potassium pyrollidone carboxylic acid (PCA), propylene glycol (PGA), sodium pyrollidone carboxylic acid (PCA), sorbitol, and sucrose. Other humectants may be used for yet additional embodiments of the compositions of the present disclosure.

Suitable thickeners include, but are not limited to, polysaccharides, in particular xantham gum, guar-guar, agar-agar, alginates, carboxymethylcellulose, relatively high molecular weight polyethylene glycol mono- and diesters of fatty acids, polyacrylates, polyvinyl alcohol and polyvinylpyrrolidone, surfactants such as, for example, ethoxylated fatty acid glycerides, esters of fatty acids with polyols such as, for example, pentaerythritol or trimethylpropane, fatty alcohol ethoxylates or alkyl oligoglucosides, and electrolytes, such as sodium chloride and ammonium chloride.

The topical compositions may further comprise one or more penetrants, compounds facilitating penetration of active ingredients into the skin of a patient. Non-limiting examples of suitable penetrants include isopropanol, polyoxyethylene ethers, terpenes, cis-fatty acids (oleic acid, palmitoleic acid), acetone, laurocapram dimethyl sulfoxide, 2-pyrrolidone, oleyl alcohol, glyceryl-3-stearate, cholesterol, myristic acid isopropyl ester, and propylene glycol. Additionally, the compositions may include surfactants or emulsifiers for forming emulsions. Either a water-in-oil or oil-in-water emulsion may be formulated. Examples of suitable emulsifiers include, but are not limited to, stearic acid, cetyl alcohol, PEG-100, stearate and glyceryl stearate, cetearyl glucoside, polysorbate 20, methylcellulose, sodium carboxymethylcellulose, glycerin, bentonite, ceteareth-20, cetyl alcohol, cetearyl alcohol, lanolin alcohol, riconyl alcohol, self-emulsifying wax (e.g., Lipowax P), cetyl palmitate, stearyl alcohol, lecithin, hydrogenated lecithin, steareth-2, steareth-20, and polyglyceryl-2 stearate.

In some formulations, such as in aerosol form, the composition may also include a propellant. For example, hydrofluoroalkanes (HFA) such as either HFA 134a (1,1,1,2-tetrafluoroethane) or HFA 227 (1,1,1,2,3,3,3-heptafluoropropane) or combinations of the two, may be used since they are widely used in medical applications. Other suitable propellants include, but are not limited to, mixtures of volatile hydrocarbons, typically propane, n-butane and isobutane, dimethyl ether (DME), methylethyl ether, nitrous oxide, and carbon dioxide. Those skilled in the art will readily appreciate that emollients, solvents, thickeners, humectants, penetrants, surfactants or emulsifiers, and propellants, other than those listed may also be employed.

When a therapeutically effective amount of the composition(s) is administered orally, it may be in the form of a solid or liquid preparation such as capsules, pills, tablets, lozenges, melts, powders, suspensions, solutions, elixirs or emulsions. Solid unit dosage forms can be capsules of the ordinary gelatin type containing, for example, surfactants, lubricants, and inert fillers such as lactose, sucrose, and cornstarch, or the dosage forms can be sustained release preparations. The pharmaceutical composition(s) may contain a solid carrier, such as a gelatin or an adjuvant. The tablet, capsule, and powder may contain from about 0.05 to about 95% of the active substance compound by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition(s) may further contain physiological saline solution, dextrose or other saccharide solution, or glycols such as ethylene glycol, propylene glycol, or polyethylene glycol. When administered in liquid form, the pharmaceutical composition(s) particularly contains from about 0.005 to about 95% by weight of the active substance. For example, a dose of about 10 mg to about 1000 mg once or twice a day could be administered orally.

In another embodiment, the composition(s) of the present disclosure can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the composition(s) in an aqueous or non-aqueous pharmaceutically acceptable solvent which may also contain suspending agents, sweetening agents, flavoring agents, and preservative agents as are known in the art.

For parenteral administration, for example, the composition(s) may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives and buffers as are known in the art.

When a therapeutically effective amount of the composition(s) is administered by intravenous, cutaneous, or subcutaneous injection, the compound is particularly in the form of a pyrogen-free, parenterally acceptable aqueous solution or suspension. The preparation of such parenterally acceptable solutions, having due regard to pH, isotonicity, stability, and the like, is well within the skill in the art. A particular pharmaceutical composition for intravenous, cutaneous, or subcutaneous injection may contain, in addition to the active agent(s), an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition(s) of the present disclosure may also contain stabilizers, preservatives, buffers, antioxidants, or other additives known to those of skill in the art.

As noted, particular amounts and modes of administration can be determined by one skilled in the art. One skilled in the art of preparing formulations can readily select the proper form and mode of administration, depending upon the particular characteristics of the composition(s) selected, the infection to be treated, the stage of the infection, and other relevant circumstances using formulation technology known in the art, described, for example, in Remington: The Science and Practice of Pharmacy, 22^{nd} ed.

Additional pharmaceutical methods may be employed to control the duration of action of the composition(s). Increased half-life and/or controlled release preparations may be achieved through the use of polymers to conjugate, complex with, and/or absorb the active substances described herein. The controlled delivery and/or increased half-life may be achieved by selecting appropriate macromolecules (for example but not by way of limitation, polysaccharides, polyesters, polyamino acids, homopolymers polyvinyl pyrrolidone, ethylenevinylacetate, methylcellulose, or carboxymethylcellulose, and acrylamides such as N-(2-hydroxypropyl) methacrylamide), and the appropriate concentration of macromolecules as well as the methods of incorporation, in order to control release. The compound(s) may also be ionically or covalently conjugated to the macromolecules described above.

Another possible method useful in controlling the duration of action of the composition(s) by controlled release preparations and half-life is incorporation of the composition(s) or functional derivatives thereof into particles of a polymeric material such as polyesters, polyamides, polyamino acids, hydrogels, poly(lactic acid), ethylene vinylacetate copolymers, copolymer micelles of, for example, PEG and poly(l-aspartamide).

Examples of bacterial families which contain bacterial species against which the presently disclosed compositions and treatment protocols are effective include, but are not limited to: Alicyclobacillaceae, Bacillaceae, Listeriaceae, Paenibacillaceae, Pasteuriaceae, Planococcaceae, Sporolactobacillaceae, Pseudomonadaceae, Staphylococcaceae, Thermoactinomycetaceae, Aerococcaceae, Carnobacteriaceae, Enterobacteriaceae, Enterococcaceae, Lactobacillaceae, Leuconostocaceae, Streptococcaceae, Caldicoprobacteraceae, Christensenellaceae, Clostridiaceae, Defluviitaleaceae, Eubacteriaceae, Graciibacteraceae, Heliobacteriaceae, Lachnospiraceae, Oscillospiraceae, Peptococcaceae, Peptostreptococcaceae, Ruminococcaceae, Syntrophomonadaceae, Veillonellaceae, Halanaerobiaceae, Halobacteroidaceae, Natranaerobiaceae, Thermoanaerobacteraceae, and Thermodesulfobiaceae.

Specific bacteria that can be treated with the compositions and methods of the present disclosure include, but are not limited to: Staphylococcus aureus, methicillin-resistant Staphylococcus aureus (MRSA), oxacillin-resistant Staphylococcus aureus (ORSA), vancomycin-resistant Staphylococcus aureus (VRSA), Staphylococcus epidermidis, methicillin-resistant Staphylococcus epidermidis (MRSE), Enterococcus faecalis, Enterococcus faecium, a Streptococcus pneumonia, penicillin-resistant Streptococcus pneumonia, Streptococcus mutans, Streptococcus sanguinis, Streptococcus viridans, Bacillus anthracis, Bacillus cereus, Clostridium botulinum, Listeria monocytogenes, Klebsiella pneumoniae, Klebsiella oxytoca, Klebsiella sp., Escherichia coli, pathogenic Escherichia coli, Pseudomonas aeruginosa, multidrug-resistant Pseudomonas aeruginosa, Salmonella sp., Salmonella enterica, Shigella sp., Carbapenem-resistant Enterobacteriaceae (CRE), and extended-spectrum beta lactamase (ESBL) positive bacteria (e.g., E. coli and Enterobacter sp.).

In certain embodiments, the compositions of the present disclosure may be provided as a package or kit, which include, for example, substantially pure preparations of the active agents described herein, combined with pharmaceutically acceptable carriers, diluents, solvents, excipients, and/or vehicles to produce an appropriate pharmaceutical composition. One embodiment of such a package or kit therefore includes at least one container with an antibiotic and at least one container with a potentiating compound. One embodiment of such a package or kit therefore includes at least one container with an antibiotic and at least one container with a BPEI compound and optionally at least one container with a β-lactamase inhibitor. Each container may comprise a pharmaceutically acceptable carrier, diluent, solvent, excipient, and/or vehicle. Each container may comprise one or more doses of the antibiotic and/or of the BPEI compound and/or of the β-lactamase inhibitor. The package or kit may comprise a plurality of containers with an antibiotic, BPEI compound, and/or β-lactamase inhibitor. The package or kit may comprise a plurality of containers each with the same antibiotic or with a different a antibiotic, a plurality of containers each with the same BPEI compound or different BPEI compounds, and/or a plurality of containers each with the same β-lactamase inhibitor or different β-lactamase inhibitors. The package or kit may further comprise a set of directions for administering the antibiotic(s), potentiating compound(s), and/or β-lactamase inhibitor(s).

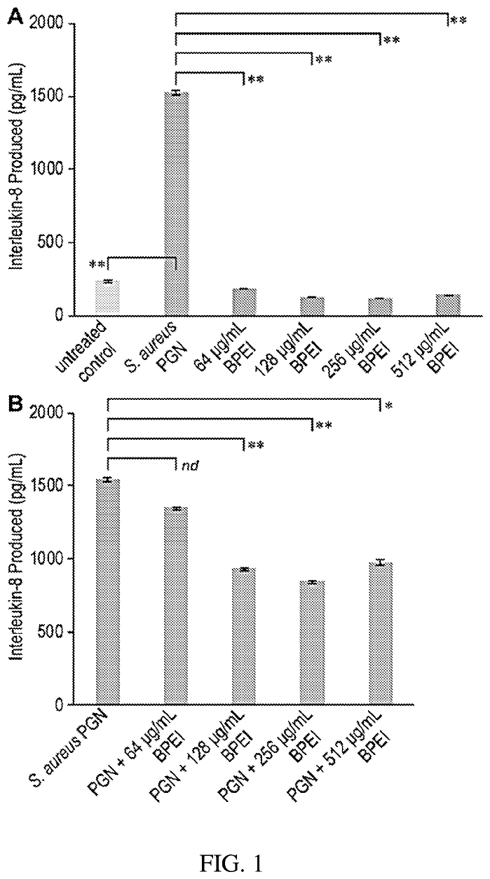

In exemplary, non-limiting embodiments of the present disclosure, 600 Da BPEI or a PEGylated 600 Da restores CRE susceptibility to carbapenem antibiotics even in the absence of a BLI such as vaborbactam, relebactam, avibactam, or tazobactam. In certain embodiments, the CRE bacterium is a K. pneumoniae strain (e.g., ATCC BAA-2146) harboring New-Delhi MBL (NDM-1) or an E. coli strain with NDM-1 (e.g., ATCC BAA-2452), or other CRE bacterial strains, for example those described elsewhere herein. As noted, the BPEI may be PEGylated, in one non-limiting embodiment, with a single PEG chain, such as a 350 MW polyethylene glycol (PEG350), and the carbapenem is meropenem (MER) or imipenem (IMI). Without the BPEI as a potentiator, the NDM-1 strains are resistant to MER and IMI because their minimum inhibitory concentrations (MIC) are over the susceptibility breakpoints of 8 and 2 μg/mL, respectively (FIGS. 1A-1H). However, 16 μg/mL (or greater) of either potentiator (BPEI or PEG-BPEI) reduces the MIC to values below the resistance breakpoints. In certain embodiments of the present disclosure, a β-lactamase inhibitor, such as vaborbactam, tazobactam, or relebactam may be used in addition to the carbapenem antibiotic and BPEI or PEG-BPEI to enhance restoration of susceptibility of the CRE bacterium to the carbapenem antibiotic. Conjugation of the PEG to the BPEI lowers toxicity of the BPEI but does not prevent the ability of the BPEI to potentiate the effect of the carbapenem antibiotic against CRE bacteria, such as those of E. coli and K. pneumoniae, and others described elsewhere herein.

### EXAMPLES

The inventive concepts of the present disclosure will now be discussed in terms of several specific, non-limiting, examples. The examples described below, which include particular embodiments, will serve to illustrate the practice of the present disclosure, it being understood that the particulars shown are by way of example and for purposes of illustrative discussion of particular embodiments of the present disclosure only and are not considered to be limiting of the claimed inventions and are presented in the cause of providing what is believed to be a useful and readily understood description of procedures as well as of the principles and conceptual aspects of the inventive concepts.

### Example 1: Anti-Inflammatory Effects of BPEI and PEG-BPEI

Each year, millions of acute skin and soft tissue infections (SSTIs) become chronic wound infections. Instead of taking 3-6 weeks to heal, chronic wounds can persist for 3-6 months. Delays in healing acute SSTIs are often due to a prolonged inflammatory phase of healing caused by bacterial debris, such as lipopolysaccharide (LPS), lipoteichoic acid (LTA), and peptidoglycan (PG). These are Pathogen-Associated Molecular Pattern molecules (PAMPs). Preventing PAMPs from triggering the release of inflammatory cytokines can restore the optimal inflammatory response. However, drugs which can successfully inhibit the release of inflammatory cytokines has been an elusive goal because the inflammatory debris originates from many different species of Gram-negative and Gram-positive bacteria. Therefore, there exists a need for a broad-spectrum therapeutic agent against LPS, LTA, and PG bacterial PAMPs. Without this agent, many acute infections will needlessly become chronic wounds, which increases the risk of recurrent infection and tissue necrosis, resulting in substantial morbidity, disability, hospitalization, and mortality, especially among older adults.

Work presented herein demonstrates that 600-Da BPEI, and its Pegylated versions (PEG-BPEIs) have activity as a broad-spectrum anti-inflammatory agent. Without wishing to be bound by theory, cationic amines of the 600-Da BPEI bind with anionic LPS and LTA (e.g., from E. coli, P. aeruginosa, and S. aureus), which reduces the ability of these agents to cause cytokine release. This behavior differs from that of monoclonal antibodies that use hydrophobic interactions to bind with polysaccharides and/or hydrophobic acyl tails of LPS and LTA, which leads to extensive variability in the ability of antibodies to target PAMPs from different bacterial strains. Following the antibody paradigm, BPEI binding to LPS, LTA, and PG will prevent their recognition by receptors. 600-Da BPEI disrupts biofilms, whereas antibodies cannot disrupt biofilms. Favorable skin tolerance and biocompatibility of 600-Da BPEI has been reported. PEG-BPEIs have lower in vivo toxicity. In a mouse model, the maximum tolerable dose, MTD, is 75-200 mg/kg for PEG-BPEIs whereas the MTD=25 mg/kg for 600-Da BPEI. Thus, although amine-based drugs are known for toxicity and off-target effects, PEGylation alleviates these problems

### Methods

Materials included HEKa cells (primary human epithelial keratinocytes; Invitrogen), Epilife Medium and growth supplement (Invitrogen), lipopolysaccharides from Escherichia coli O111:B4 (Sigma), peptidoglycan from Staphylococcus aureus (Sigma), and Human (IL-8/CXCL8, IL-6, TNF-alpha) Quantikine ELISA Kits (R&D). HEKa cells were seeded in T-75 tissue culture flasks with Epilife media supplemented with human keratinocyte growth supplement 100 ug/mL and 100 U/mL of pen/strep and incubated at 37° C. in a CO₂ incubator. Fresh media was replaced every 2 days. Until the cell confluence reach 80-90%, they were split into a new passage. To avoid cell senescence, all experiments were performed with cells at passage 3-7. HEKa cells were cultured in a new 24-well plate until 80-90% confluence (total V=1 mL/well). Then treatments of 600-Da BPEI (64, 128, 256, 512, and 1024 μg/mL), LPS (lipopolysaccharides from E. coli; 5 μg/mL), PG (peptidoglycan from S. aureus; 5 μg/mL), and LPS or PG+BPEI (with BPEI added first to the cultures and then LPS or PG with the same concentration of 5 μg/mL added after 5 min) were added in triplicate cultures for 24 hr. The cell media was collected in 1.5 mL Eppendorf microtubes and stored at −20° C. until ELISA assays were performed. Concentrations of IL-8, IL-6, TNF-α cytokines released into the media were quantified followed the instructions of Quantikine Colorimetric ELISA assay kits (R&D Systems). Absorbance was measured at 450 nm and 570 nm. Final corrected absorbance was the subtraction at 450 nm from the one at 570 nm.

### Results

Bacterial products, such as cell wall components of LPS or PG, stimulate recruitment and activation of phagocytes. These foreign products are grouped into pathogen-associated molecular patterns (PAMPs). PAMPs are recognized by toll-like receptors (TLRs) which eventually leads to secretion of cytokines IL-6, IL-8, and TNF-α in response to inflammation reactions. Large scale production of these inappropriate cytokines may cause even more diseases, including but not limited to autoimmune diseases (i.e. rheumatoid arthritis and systemic lupus erythematosus), chronic wound infections, and autoinflammatory diseases (asthma and arthritis). Therefore, a good topical anti-bacterial agent must not induce inflammation itself.

In the present work, it was found that inflammatory responses of human epithelial keratinocyte (HEKa) cells (release of IL-8, IL-6, and TNF-α) after being exposed to 600-Da BPEI for 24 hours were minimal.

Results in FIG. 1 show that S. aureus peptidoglycan (PGN) causes the release of interleukin-8 (IL-8) from primary human epithelial keratinocytes (HEKa) cells. IL-8 is a cytokine and chemokine molecule involved with neutrophil recruitment to the wound site. Its release is stimulated when peptidoglycan binds to, and is mainly recognized by, toll-like receptor 2 (TLR2). In contrast, 600-Da BPEI does not cause the HEKa cells to release IL-8 (FIG. 1a). When 600-Da BPEI and S. aureus PGN are added together to HEKa cells, the amount of IL-8 released is diminished (FIG. 1B), indicating, for example, a therapeutic benefit of BPEI for wound care.

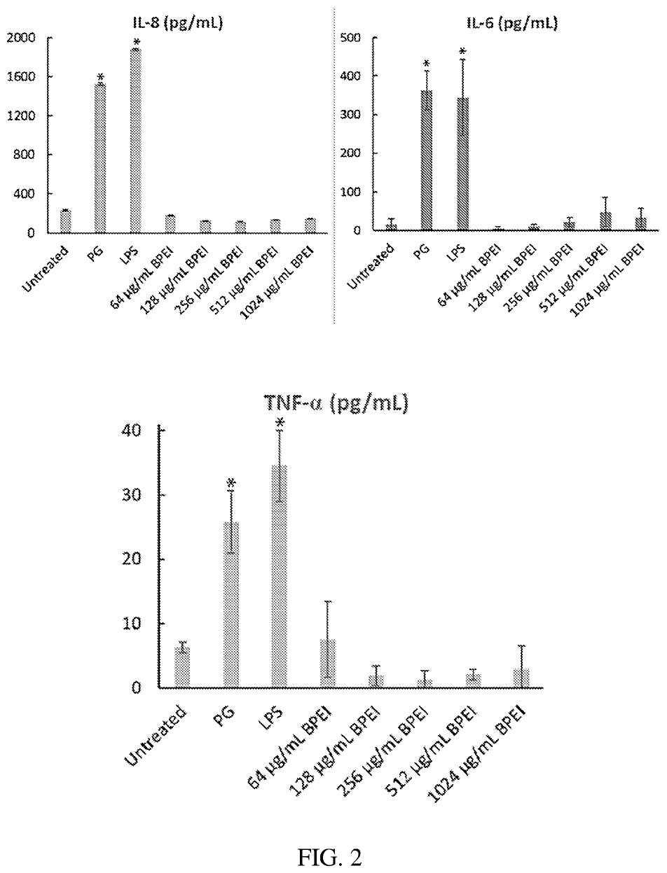

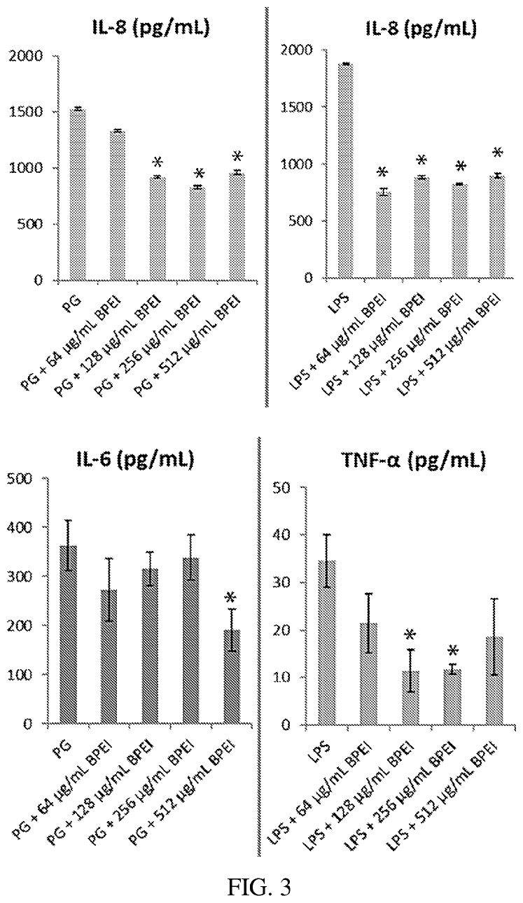

FIG. 2 shows that BPEI (at all concentrations of BPEI tested) does not induce cytokine release in HEKa cells, while PG (from S. aureus) and LPS (from E. coli) cause significant release of IL-8, IL-6, and TNF-alpha. Variety of BPEI concentrations (64-1024 μg/mL) showed no significant changes in the three cytokine responses to the untreated control. Interleukin IL-8 was found to have the highest concentrations released (>1500 pg/mL by PG/LPS and ˜200 pg/mL in untreated control). IL-6 was released in smaller amounts (˜350 pg/mL by PG/LPS and <50 pg/mL in untreated control). The cytokine released in the lowest concentration was TNF-α (˜30 pg/mL by PG/LPS and <10 pg/mL in untreated control). Larger error bars in the IL-6 and TNF-α graphs were due to the smaller in absorbance intensity that was close to a detection limit. Nevertheless, statistical analyses using t-test (95% confidence) indicate significant differences of these cytokines' release by only the bacterial cell wall components (PG and LPS), and not by 600-Da BPEI. The results shown in FIG. 3 indicate that BPEI suppresses the cytokine release caused by LPS and PG, with a greater effect on release caused by LPS.

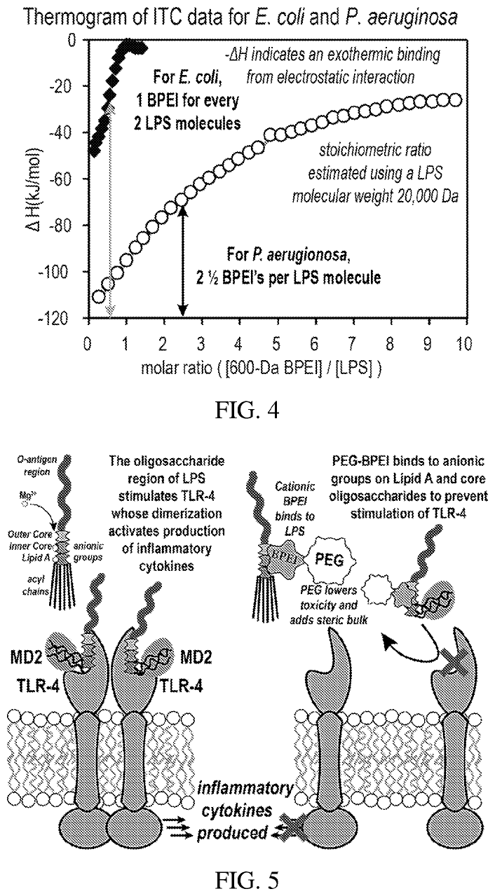

Results in shown in FIG. 4 demonstrate that 600-Da BPEI binds to LPS. Isothermal Titration calorimetry (ITC) directly measures the enthalpy of molecular binding interactions. Here, it was used to confirm interactions between BPEI and LPS. The isotherm obtained from mixing BPEI with P. aeruginosa LPS (Sigma #L8643) is shown in FIG. 4. The ITC isotherm of 600-Da BPEI binding to LPS from P. aeruginosa shows that 2.5 BPEI molecules bind with one LPS molecule, while just one molecule of BPEI binds to two molecules of LPS from E. coli, indicating a weaker interaction between 600-Da BPEI and E. coli LPS. Negative ΔH values indicate exothermic binding. This is due to electrostatic attractions between cationic BPEI and the anionic lipid A, inner-core, and outer-core oligosaccharide chains, illustrated in FIG. 5. The ITC data obtained from mixing 600-Da BPEI with E. coli LPS (InvivoGen #tlrl-3pelps) is also shown in FIG. 4. The negative ΔH values indicate exothermic binding, but 600-Da BPEI interacts with fewer E. coli LPS molecules than P. aeruginosa LPS; and less heat is released. FIG. 5 shows a possible mechanism of action for BPEI. Here, LPS leads to cytokine production when it stimulates TLR-4 (left). However, this process is interrupted when BPEI binds with LPS to prevent its recognition by receptors (right).

The bacterial endotoxin LPS is a PAMP molecule that triggers the release of inflammatory cytokines and prolongs the inflammatory phase of healing. Endotoxins, which are a type of pyrogen, are lipopolysaccharides present in the cell walls of gram-negative bacteria. Pyrogens as a class are fever-inducing substances that can be harmful or even fatal if administered to humans above certain concentrations. LPS activates a proenzyme in the Limulus amebocyte lysate (LAL), which it in turn splits a colorless substrate into the colored product para-nitroanilide. The colored product can be analyzed at 405 nm using spectrophotometry. The reason the bacterial endotoxin test is also called LAL or limulus amebocyte lysate testing is because the lysate from blood cells (amebocytes) from horseshoe crabs (Limulus polyphemus). The lysate from the horseshoe crabs blood cells reacts with bacterial endotoxins as part of the crab's immune response to the LPS endotoxin.

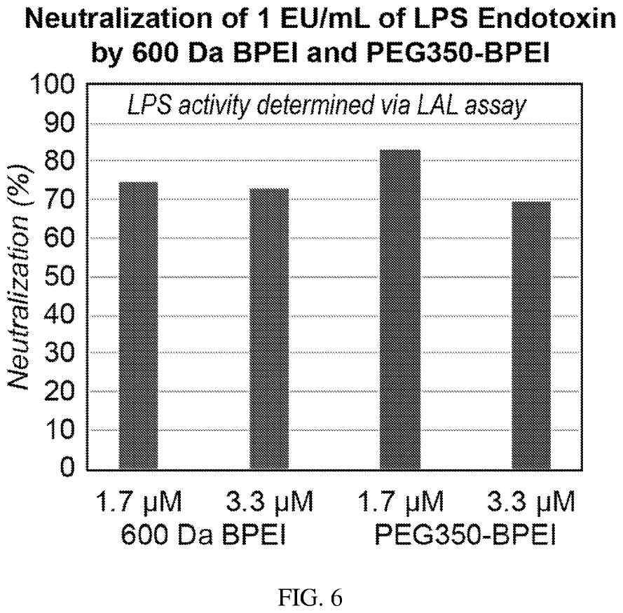

As shown in FIG. 6, both 600 Da BPEI (BPEI₆₀₀) and PEG₃₅₀-BPEI₆₀₀ neutralize LPS activity. Using a LAL assay kit, free LPS in pyrogen-free water was quantified and its neutralization shown using BPEI and PEG-BPEI. As expected, the amount of detected colored product produced by free LPS decreased after treating the solution with 600 Da BPEI or PEG350-BPEI. Using LPS from E. coli 0111:B4, we quantified free and bound LPS in pyrogen-free water. However, a sample of LPS+PEG350-BPEI resulted in a lower amount of detected colored product. These data were used to calculate the percent neutralization. PEG350-BPEI (1.7 nM) neutralized 81% of a 1 EU/mL (5 pM) LPS solution. Both BPEI and PEG-BPEI can neutralize the immune response to LPS endotoxin.

The results presented hereinabove demonstrates that low molecular weight branched polyethylenimine (600 Da BPEI and PEG350-BPEI) can function as a broad-spectrum anti-inflammatory agent in epithelial cells. The lipid A region of LPS, responsible for its immune stimulatory activity, contains anionic phosphate groups. In addition, LPS has anionic phosphates and carboxylates on its core oligosaccharides. Without wishing to be bound by theory, it is believed that when cationic BPEI binds with anionic LPS, the structural and/or conformational nature of lipid A and the core oligosaccharides is altered by the additional steric bulk of 600-Da BPEI, rendering the LPS unable to stimulate Toll-like receptor 4 (TLR-4).

Wound healing and re-establishment of an intact skin barrier is a complex process involving the response of several cell types, including fibroblasts and keratinocytes. Slow wound healing is linked to continued stimulation of the inflammasome and cytokine release from PAMPs in wounds. TLR4 primarily recognizes, and is activated by, LPS from Gram-negative bacteria. TLR4 activation requires interaction with a number of co-receptors including LPS-binding protein (LBP), CD14, and myeloid differentiation protein 2 (MD-2) to bind LPS and induce a signaling cascade. Ultimately, this leads to the activation of NF-κB and the production of pro-inflammatory cytokines. Variation in lipid A, specifically the fatty acyl chains, among diverse bacterial species cause a vast difference in the biological activity of LPS. Hexa-acylated LPS (6 fatty acid chains) is a highly active agonist of TLR4 and is found commonly on pathogenic bacteria. Under-acylated LPS (4-5 fatty acid chains) induces a significantly lower host response. Toll-like receptor 2 (TLR2) recognizes LTA, a major immune stimulatory component of Gram-positive bacteria, that causes cells to produce TNF-α and other inflammatory cytokines. PGN is an activator of TNF-α through TLR2. Electrostatic interactions between cationic BPEI and anionic LPS can prevent LPS-induced immune response.

BPEI can be used to expand the uses of certain antibiotics for therapeutic treatment of infections. As shown in the present example, BPEI can also be used to treat chronic wound infections (e.g., those that have not proceeded through a reparative process in 3 weeks-months), which are often caused by drug resistant bacteria, such as MRSA and MDR-PA. In one embodiment of the present disclosure, BPEI and PEG-BPEI can be used topically orally or intravenously or by inhalation, as a wound treatment to disable biofilms, resistance mechanisms, and reduce inflammation in the tissues of the wound, or to inhibit inflammation caused by bacteria. The BPEI causes a lowering of the release of inflammation-causing cytokines in response to peptidoglycan stimulation. Further, reducing inflammation can prevent many acute infections from becoming chronic wounds; and lowers the risk of recurrent infection and tissue necrosis that results in substantial morbidity, disability, hospitalization, and mortality, especially among older adults.

In other embodiments, the BPEI compounds of the present disclosure can be used to reduce inflammation in lung epithelial tissues such as bronchial/tracheal epithelial cells and small airway epithelial cells. Examples of lung epithelial cells that can be used in vitro to show such an effect include, but are not limited to, the cell lines ATCC® PCS-300-010 and ATCC® PCS-301-010. In one non-limiting embodiment, the BPEI compounds of the present disclosure can be used in treatments for COVID-19. In certain embodiments, the BPEI compounds of the present disclosure can be used in treatments for pathogenic airway inflammation, asthma, pulmonary fibrosis, chronic obstructive pulmonary disease, chronic bronchitis, and microbial lung infections including pneumonia.

In other embodiments, the BPEI compounds of the present disclosure can be used to reduce inflammation in intestinal epithelial tissues such as small intestine or colon epithelial cells for the treatment of intestinal disease having an inflammatory component such as ulcerative colitis and IBD. Examples of intestinal epithelial cells that can be used in vitro to show such an effect include, but are not limited to, the cell lines ATCC® CCl-241™, CRL-3266™ (small intestine) or ATCC® CRl-1459™, CRL-1539™, CRL-1831™ (colon). In other embodiments, the BPEI compounds of the present disclosure can be used as treatments for septic shock.

### Example 2: Overcoming Resistance in Carbapenem-Resistant Enterobacteriaceae (CRE) Bacteria

### Methods

CRE expressing the metallo-β-lactamase NDM-1, E. coli BAA-2452™ and K. pneumoniae BAA-2146™, were purchased from the American Type Culture Collection (ATCC). Chemicals were purchased from Sigma-Aldrich, Inc. and antibiotics obtained from GoldBio, Inc. 600 Da BPEI (BPEI₆₀₀) was purchased from Polysciences, Inc. PEG-BPEI, a product of the combination of PEG₃₅₀ and BPEI₆₀₀, was synthesized as described elsewhere herein. Briefly, monofunctionalized PEG-epoxide was obtained from Nanocs, Inc. Approximately 600 Da BPEI was dried overnight with lyophilization and the final mass used to determine amount of mPEG-epoxide (350 MW) required to react with 600 Da BPEI in a 1-to-1 stoichiometric ratio. Both 600 Da BPEI and mPEG-epoxide are dispersed in anhydrous ethanol and at 60° C. for 24 hours. The success of the epoxide ring-opening reactions was determined by ¹H NMR spectroscopy.

Checkerboard Assays

Checkerboard assays were used to determine the synergistic effect between PEG350-BPEI or 600 Da BPEI and antibiotics against drug resistant strains growing in cation-adjusted Mueller-Hinton broth (CAMHB). Bacterial growth used CAMHB media augmented with various amounts in serial dilutions of potentiator and antibiotic (oxacillin or piperacillin) inoculated with bacterial cells from an overnight culture (5×10⁵ CFU/mL). Cells were grown at 37° C. The change in OD₆₀₀ (optical density at 600 nm) was measured and recorded after 24 hr of treatment. Each checkerboard trial was done in triplicate using sterile Greiner CellStar™ flat bottom polystyrene plates, catalog #655180.

Isothermal Titration calorimetry (ITC)

Isothermal titration calorimetry (MicroCal PEAQ-ITC, Malvern Inc., Malvern, U.K.) was utilized to quantify the heat release upon mixing 600 Da BPEI with ZnCl₂ and PEG350-BPEI with ZnCl₂. Briefly, solutions of 600 Da BPEI or PEG350-BPEI (0.105 mM) and 1.8 mM ZnCl₂ were prepared in 50 mM Tris-HCl (pH 7) were titrated using injections of 2 μL lasting 4 s and separated by 150 s time intervals. Controls were performed and the experiment was done in duplicate.

H33342 Bisbenzimide Dye Uptake Assay

Overnight culture of E. coli BAA-2452™ and K. pneumoniae BAA-2146™ were used to inoculate fresh CAMHB media for another 5 hr at 35° C. with shaking. Bacterial cells were collected by centrifugation at 6000 rpm for 40 min and resuspended in PBS. The OD₆₀₀ of the cell suspension was adjusted to about 1.0 and kept at room temperature during the experiment. Aliquots (180 μL/well) of the cell suspension were transferred to a 96-well flat-bottom black plate in the format of column 1, PBS blank; column 2, untreated control cells; column 3, cells+potentiator (sub-lethal concentration). Five technical replicates of each group were conducted. Fluorescent probe Hoechst 33342 bisbenzimide (H33342) was added (20 μL) to each well with a final concentration of 5 μM. Fluorescence was read immediately after the addition of H33342 by a Tecan Infinite M20 plate reader with the excitation and emission filters of 355 and 460 nm for H33342. Fluorescence data were normalized to the emission before cells were added in the PBS control, and they were plotted against time to show the cellular uptake of H33342 over 10 min. Control experiments of dye+600-Da BPEI were unchanged from fluorescence emission values obtained with dye only.

### Results

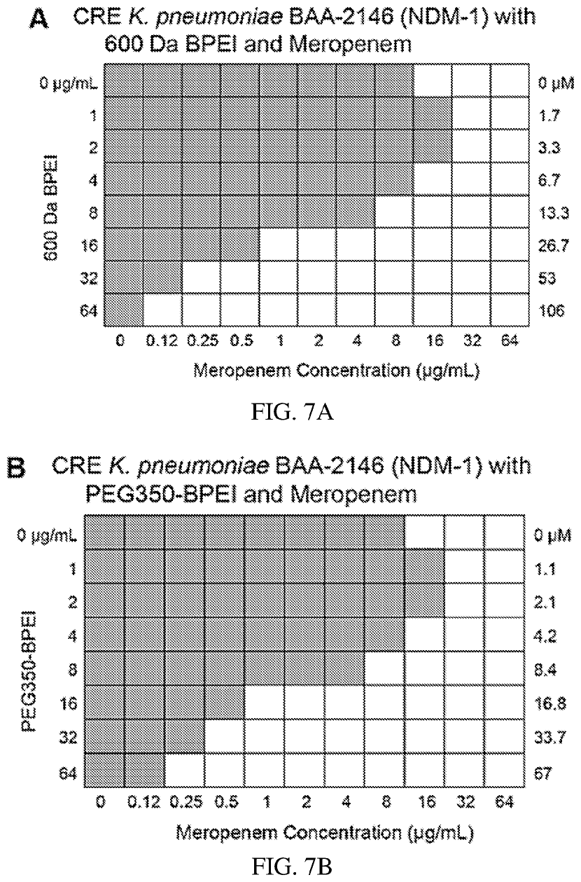

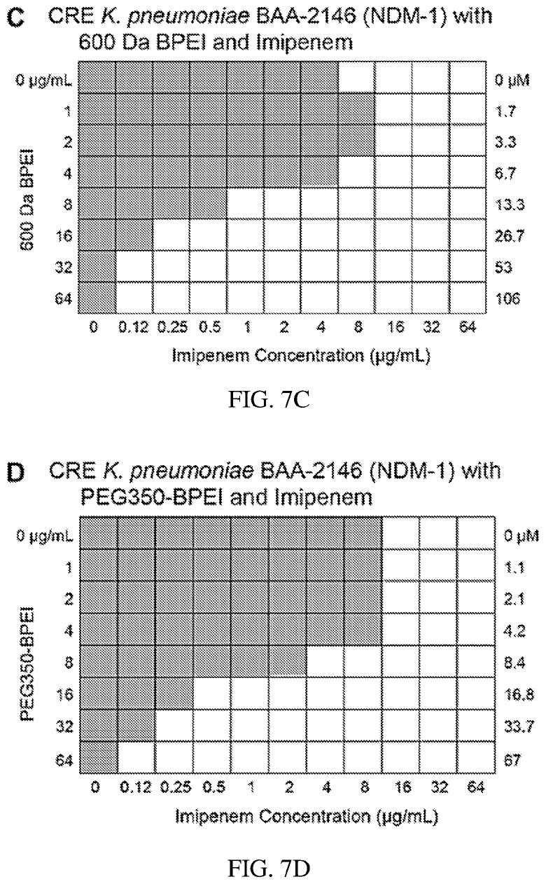

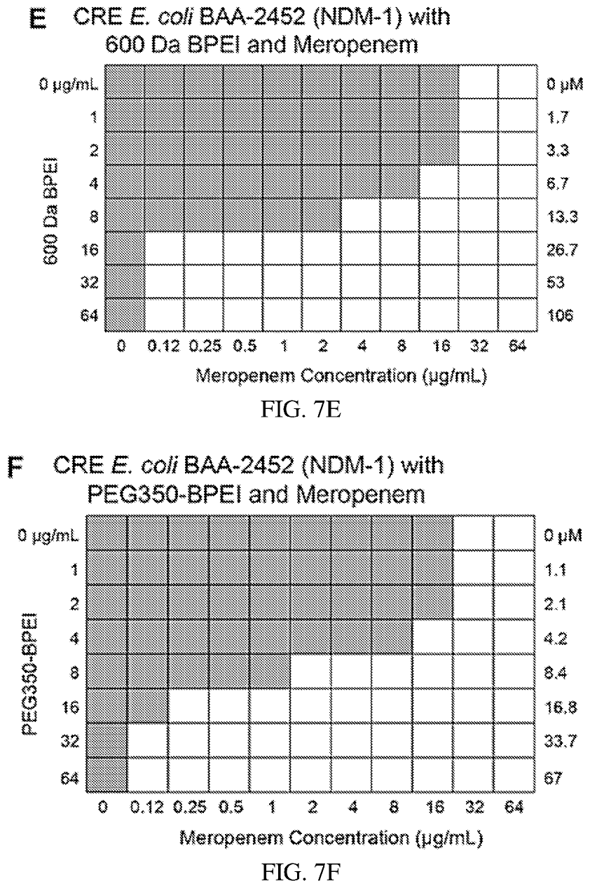

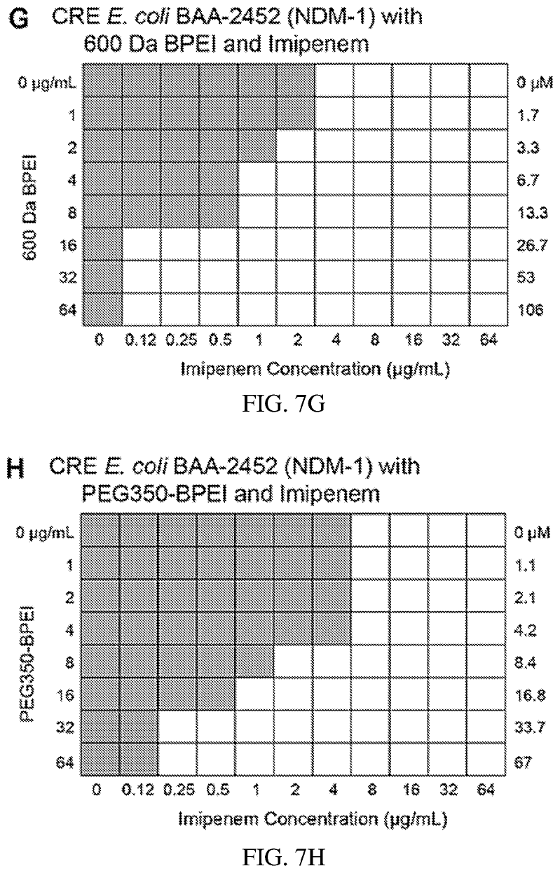

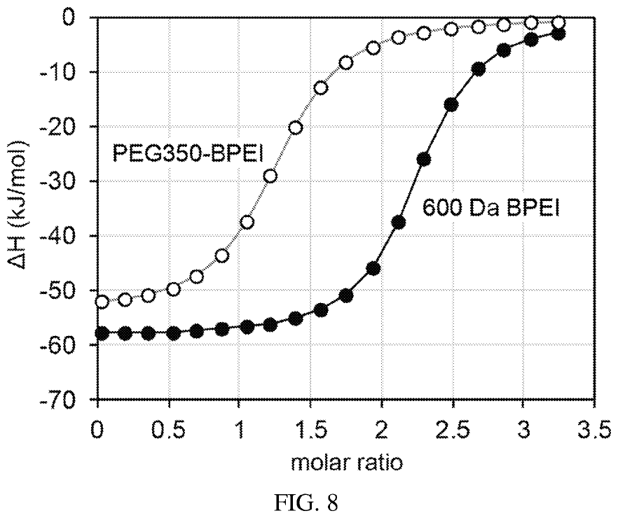

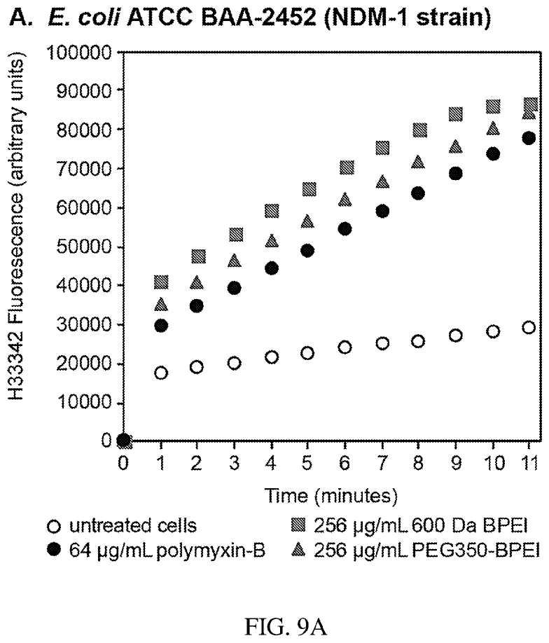

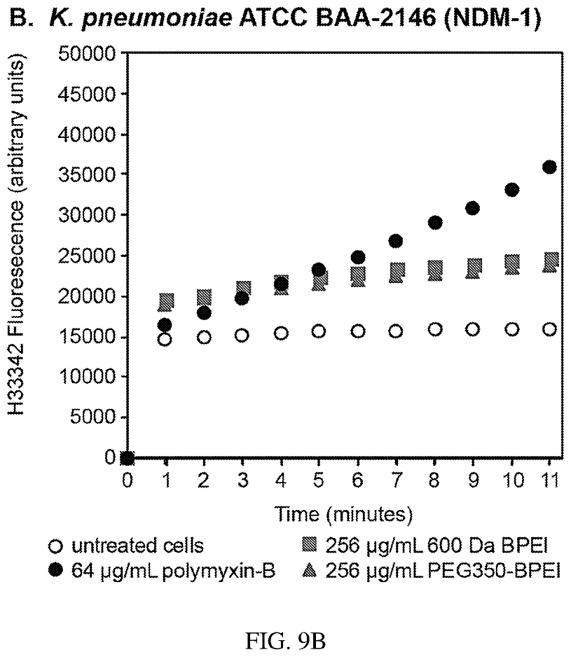

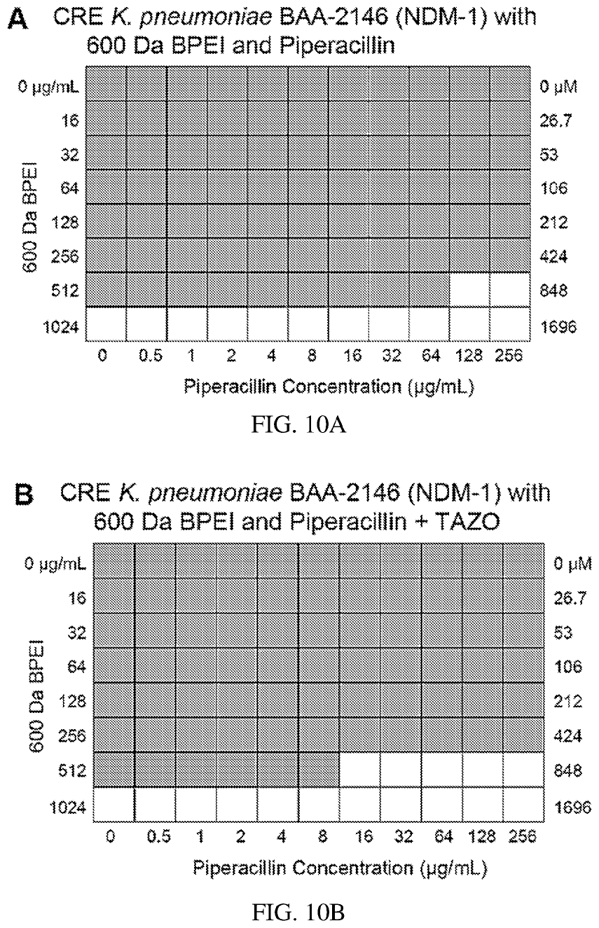

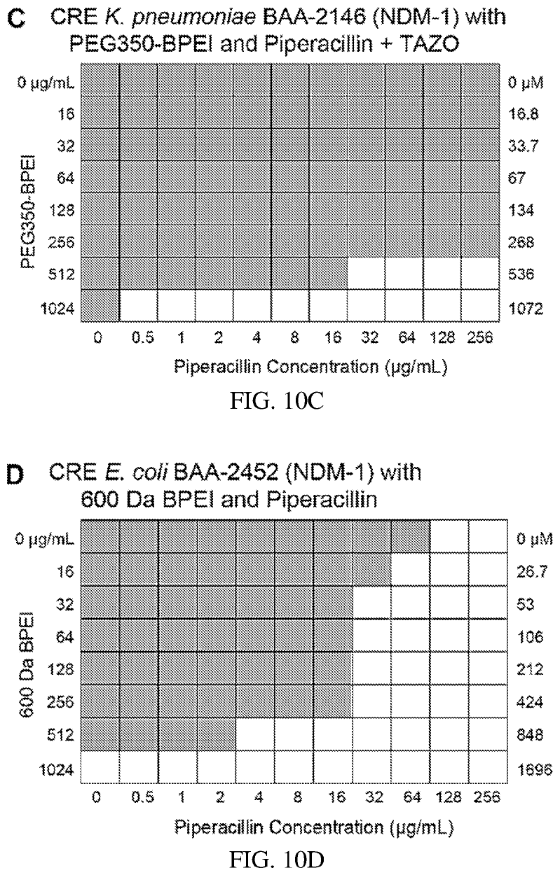

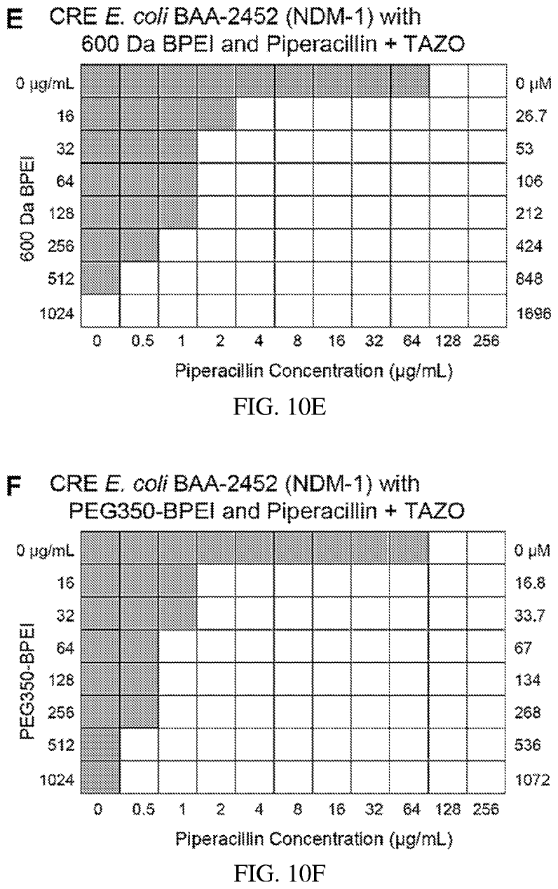

The NDM-1 enzyme requires Zn²⁺ ions for activity. Overcoming carbapenem resistance by disabling MBLs with Zn²⁺ chelators is well-known. Other strategies overcome resistance by targeting MBLs with inhibitors. Compounds that bind to the LPS layer to facilitate the influx of antibacterial agents are often designed from peptide or peptidomimetic scaffolds although resistance has developed. These well-founded and important developments highlight the utility of single-function compounds that inhibit carbapenemases, inhibit efflux pumps, increase the uptake of these inhibitors and various antibiotics, or chelate and sequester Zn²⁺ ions essential for MBLs. The amines of PEG-BPEI and 600 Da BPEI can chelate metals. 600 Da BPEI binds Cu²⁺ but does not bind Ca²⁺ or Mg²⁺. Binding between Zn²⁺ and 600 Da BPEI is known and we demonstrate this phenomenon here using isothermal titration calorimetry (ITC). Metal chelation is an exothermic process and the release of heat for Zn²⁺ binding with PEG-BPEI and 600 Da BPEI is nearly identical (FIG. 8). In addition to the chelation of essential Zn²⁺ ions, 600 Da BPEI and PEG-BPEI have other properties, such as disrupting biofilms and increasing antibiotic influx without the need to disrupt the membrane. With regard to the latter effect, we investigated the ability of 600 Da BPEI and PEG-BPEI to increase drug uptake in K. pneumoniae ATCC BAA-2146 and E. coli ATCC BAA-2452. As shown in FIG. 9A, the fluorescence of H33342 increases in the presence of 600 Da BPEI, PEG-BPEI, and polymyxin-B (PMB). The cells were harvested and studied at similar concentrations (OD₆₀₀ values adjusted to 1.0) and the effect of enhanced influx is greatest with the NDM-1 E. coli strain BAA-2452. Here, the concentrations of 600 Da BPEI and PEG-BPEI are higher than PMB, and much higher than the amount required for potentiation (FIGS. 7A-7H). As observed previously with H33342 influx against P. aeruginosa, it is possible that, at high concentrations, membrane disruption may be occurring but an important consideration is the cell density in the H33342 assay (7×10⁸ CFU/mL) is over 1000 times higher than that of the checkerboard assays (5×10⁵ CFU/mL). Thus, at the low concentration (16 μg/mL) that enables meropenem and imipenem potentiation, membrane disruption is unlikely to occur and thus the mechanism of action is chelation of Zn²⁺ ions. Pronounced differences are observed in the H33342 assay data for K. pneumoniae BAA-2146 (FIG. 9B). First, the corresponding fluorescence intensities are lower than the E. coli NDM-1 strain. Secondly, 600 Da BPEI and PEG-BPEI have a smaller effect on H33342 influx and these effects are smaller than that of PMB. The smaller influence of 600 Da BPEI and PEG350-BPEI on H33342 uptake by K. pneumoniae NDM-1 should be reflected in the antibiotic potentiation of other agents, such as the β-lactam piperacillin. Indeed, 600 Da BPEI improves piperacillin efficacy against the E. coli NDM-1 strain (FIG. 10D) but potentiation is not observed against K. pneumoniae BAA-2146 (FIG. 10A). The paradigm increasing drug uptake applies equally to increasing the influx of β-lactamase inhibitors. FIGS. 10E and 10F show that 16 μg/mL of 600 Da BPEI and PEG350-BPEI lower the piperacillin MIC to a value below its susceptibility breakpoint in the presence of 4 μg/mL of tazobactam, a β-lactamase inhibitor. However, this effect is only observed with E. coli NDM-1 and not the K. pneumoniae NDM-1 strain (FIGS. 10B-10C).

Although PEGylation of BPEI mitigates in vivo toxicity, adverse events from systemic exposure remain a concern. Thus, in at least certain embodiments, PEG-BPEIs can be used as topical agents applied to acute and chronic wounds because PEG-BPEI+antibiotic combinations can kill susceptible and resistant bacteria in their biofilm and planktonic environments. Both 600 Da BPEI and PEG-BPEI disrupt biofilms and disable resistance mechanisms of lab-strains and clinical isolates of MDR-PA, MRSA and MRSE. The data presented herein demonstrate that the utility of PEG-BPEI potentiators can be expanded to life-threatening CRE infections that are an urgent medical need. CRE bacteria produce enzymes that degrade a wide range of antibiotics, including carbapenems and β-lactams. There is a high mortality rate and survivors can have severe morbidity from treatment with toxic last-resort antibiotics. CRE have mobile genetic elements that transfer resistance genes to other species. These bacteria also circulate throughout the healthcare system. The mobility and spread of CRE needs to be curtailed, but these goals are impeded by wounds colonized and infected with CRE. CRE wound infections are often polymicrobial, facilitating gene transfer. CRE wound infections also aggravate CRE spread because patients reside in, and are transported among, out-patient clinical facilities, in-patient hospitals, long-term care facilities, long-term acute care facilities, and skilled nursing home facilities. Thus, improved therapeutic agents against CRE-infected wounds may lead to better control of CRE exposure and spread. In certain embodiments, these compounds also have added benefits of mitigating wound biofilms and inflammatory cytokines that impair healing. 600 Da BPEI provides these benefits, and modification with polyethylene glycol (PEG), forming PEG-BPEI, reduces in vivo toxicity. In a mouse model, the maximum tolerable dose, MTD, is 75 mg/kg for PEG350-BPEI whereas the MTD=25 mg/kg for 600-Da BPEI.

Without wishing to be bound by theory, it is believed that the potentiation mechanism of action (MOA) involves the chelation of Zn²⁺ ions essential for metallo-β-lactamases. PEG-BPEI can chelate Zn²⁺ ions. Binding between Zn²⁺ and 600 Da BPEI is known and shown by ITC (FIG. 8). 600 Da BPEI binds Cu²⁺ but does not bind Ca²⁺ or Mg²⁺. Additionally, PEG-BPEI can function as an influx potentiator. The core oligosaccharides have anionic phosphates that chelate Mg²⁺ ions in the LPS layer. LPS is a barrier that hinders drug influx. Cationic PEG-BPEI will bind with these anionic sites to improve diffusion through the LPS. PEG-BPEI could also bind to the phosphates on Lipid A, located near the layer of hydrophobic acyl chains. However, PEG-BPEI is hydrophilic and thus lacks the driving force to enter the hydrophobic regions. This characteristic distinguishes PEG-BPEI from cationic polymyxins and colistin, whose hydrophobic alkyl chains are required for membrane disruption. We've reported potentiation across antibiotic classes by lowering LPS barriers, which enhances passive diffusion and porin-mediated drug influx mechanisms. This paradigm applies equally to increasing the influx of β-lactamase inhibitors, such as tazobactam, and can also improve the uptake of efflux-pump inhibitors. These complementary MOAs are enhance the value of PEG-BPEI, which also disrupts biofilms, an underlying pathology that contributes to delayed healing. Wound healing will also improve because PEG-BPEI binds to Gram-positive and Gram-negative PAMP molecules to reduce the release of inflammatory cytokines.

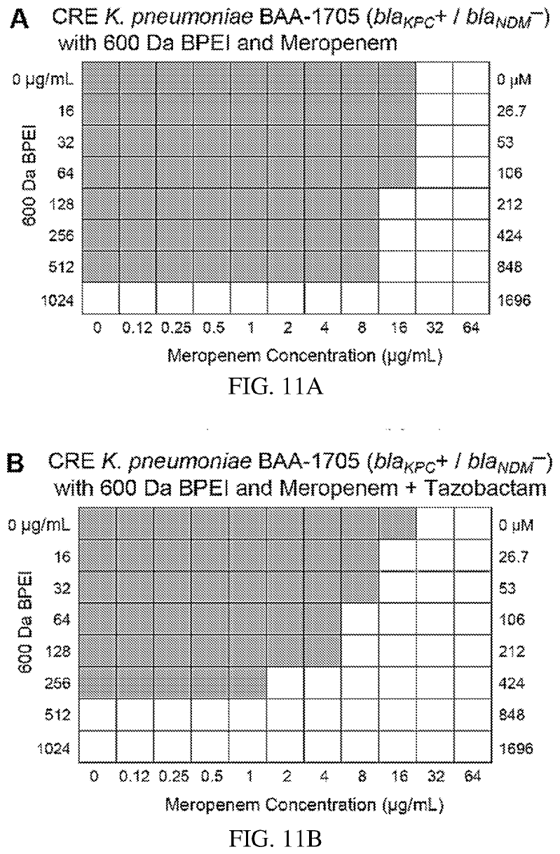

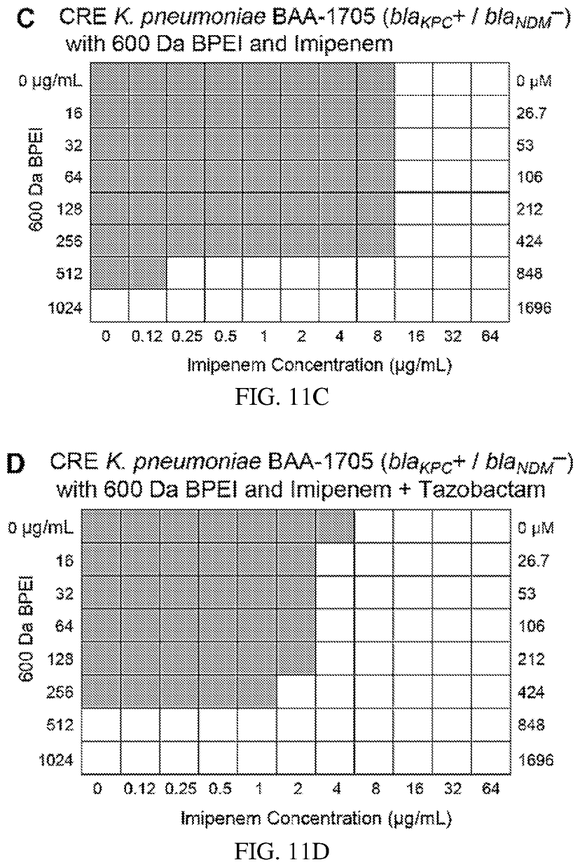

The capacity of a compound to inhibit bacterial growth is characterized by the parameter minimum inhibitory concentration (MIC) and MIC is used to define a susceptibly vs. resistance breakpoint. Against Enterobacteriaceae, the susceptibility breakpoints of MER and IMI are 8 and 2 μg/mL, respectively. As shown in FIGS. 11A and 11C, K. pneumoniae ATCC BAA-1705 is resistant to the carbapenem antibiotics meropenem (MER) and imipenem (IMI). By comparing FIG. 11A with FIG. 11B, restoring carbapenem susceptibility with 600 Da BPEI requires the use of a β-lactamase inhibitor, such as tazobactam. However, this approach does not work with combined application of IMI and tazobactam (FIG. 11D).

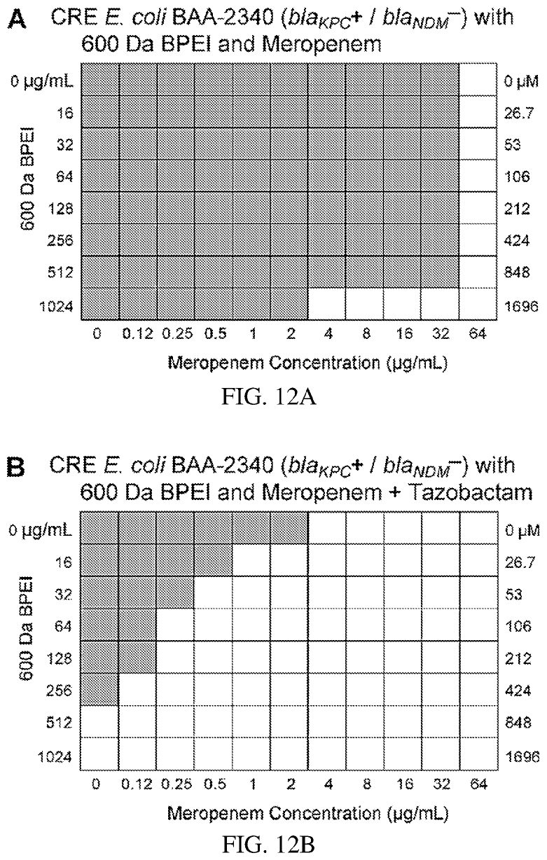

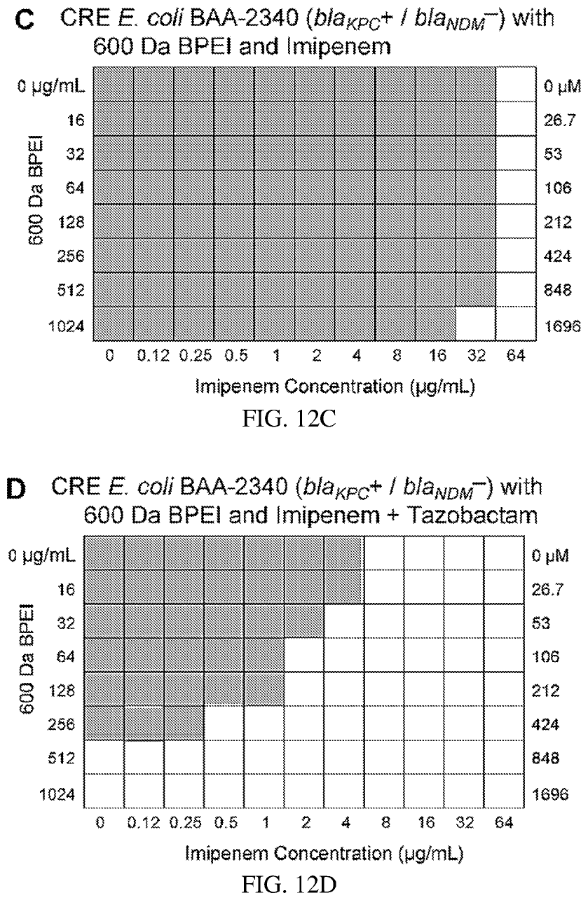

Restoring carbapenem susceptibility in E. coli BAA-2340 is more successful. As shown in FIGS. 12A and 12C, E. coli ATCC BAA-2340 is also resistant to MER+600 Da BPEI and IMI+600 Da BPEI. When a β-lactamase inhibitor is added, the checkerboard assay results show that resistance to MER and IMI is decreased, although the combination of 600 Da BPEI, meropenem, and tazobactam (FIG. 12B) is more effective than the combination of 600 Da BPEI, imipenem, and tazobactam (FIG. 12D).

### Example 3: Potentiation of Macrolide Antibiotics by BPEI Against Antibiotic-Resistant Bacteria

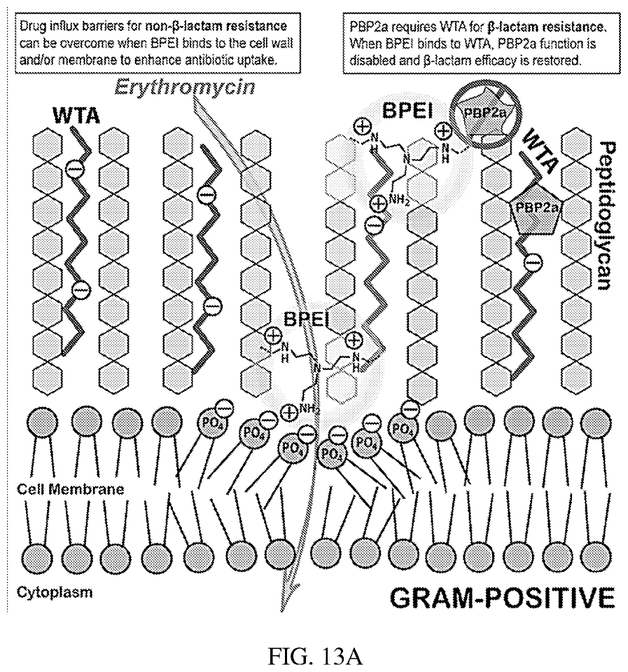

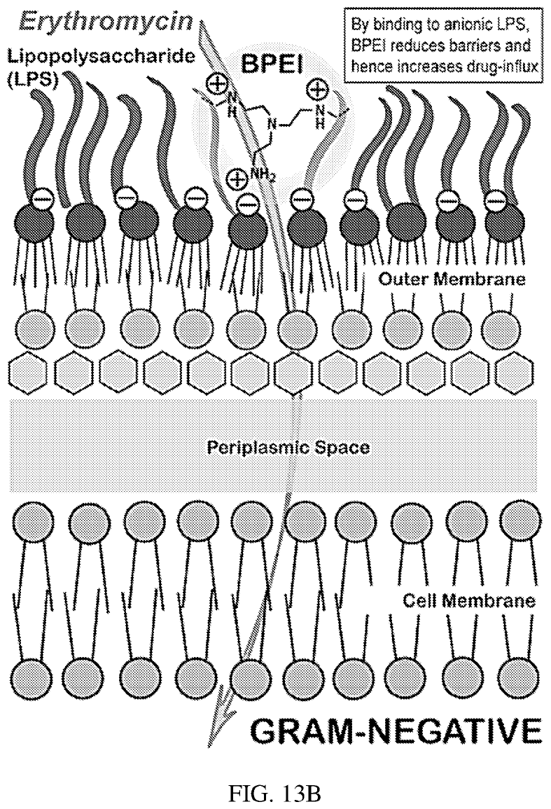

In the present work, in certain embodiments, low MW BPEI polymers (which may be pegylated) are used as antibiotic adjuvants. In particular, it is shown that clinical isolates of MRSA having erythromycin resistance can be rendered drug-susceptible when 600-Da BPEI is used to reduce the barriers to drug-influx. It is also shown that 600-Da BPEI potentiates erythromycin and azithromycin against clinical isolates of MDR-PA. This is noteworthy because erythromycin has been regarded as an antibiotic without efficacy against Gram-negative bacteria, including those without antimicrobial resistance. Without wishing to be bound by theory, it is believed that the MOA of the BPEI involves binding with anionic sites of LPS in the bacterial cell envelope, wall teichoic acid (WTA), and lipoteichoic acid (LTA)) to create new hydrophilic conduits for erythromycin to reach the cytoplasm (FIG. 13A). 600-Da BPEI is hydrophilic and targets anionic sites on the cell envelope away from the alkyl chains of membrane bilayers. It reduces diffusion barriers to increase drug uptake and enables broad-spectrum efficacy against different bacterial species. Instead of acting as an antimicrobial agent itself, low concentrations of 600-Da BPEI potentiate the efficacy of erythromycin against clinical isolates of MRSA and MDR-PA. Additionally, as discussed in further detail below, BPEI reduces cytokine release from primary human epithelial keratinocytes (HEKa) cells, indicating another therapeutic application of BPEI (with or without an antibiotic) as a treatment for wound care.

### Methods

Two MRSA clinical isolates (MRSA OU6 and MRSA OU11) and an MDR isolate of P. aeruginosa (PA OU19) from patient swabs were kindly provided by Dr. McCloskey from the University of Health Sciences Center with an institutional review board (IRB) approval. Chemicals (DMSO, growth media, erythromycin, polymyxin B, H33342 dye, and PGN from Staphylococcus aureus (product number 77140) were purchased from Sigma-Aldrich. 600-Da BPEI was purchased from Polysciences. HEKa cells (primary human epithelial keratinocytes), Epilife Medium, and growth supplement were purchased from Invitrogen. Human IL-8/CXCL8 Quantikine ELISA Kit was purchased from R&D.

The multi-drug resistance characteristics of the clinical isolates was confirmed using the Beckman Coulter MicroScan Walkaway™ 96plus with the PC33 gram positive panel. The MIC values are listed below.

MRSA OU6: oxacillin>2 μg/mL (resistant); clindamycin>4 μg/mL (resistant); daptomycin≤0.5 μg/mL (susceptible); erythromycin>4 μg/mL (resistant); gentamicin≤4 μg/mL (susceptible); linezolid 2 μg/mL (susceptible); tetracycline≤4 μg/mL (susceptible); vancomycin 2 μg/mL (susceptible).

MRSA OU11: oxacillin>2 μg/mL (resistant); clindamycin>4 μg/mL (resistant); daptomycin≤0.5 μg/mL (susceptible); erythromycin>4 μg/mL (resistant); gentamicin≤4 μg/mL (susceptible); linezolid 2 μg/mL (susceptible); tetracycline≤4 μg/mL (susceptible); vancomycin 1 μg/mL (susceptible).

PA OU19: aztreonam 16 μg/mL (resistant); cefepime 16 μg/mL (resistant); ceftazidime 16 μg/mL (resistant); ciprofloxacin 2 μg/mL (resistant); gentamicin 4 μg/mL (susceptible); meropenem 8 μg/mL (resistant); piperacillin+tazobactam 64 μg/mL (resistant); tobramycin 4 μg/mL (susceptible).

Checkerboard Assays

Checkerboard assays were conducted to identify synergy between BPEI and antibiotics against bacteria. Serial dilutions of antimicrobial agents (BPEI and antibiotic solutions) were added to a 96-well microtiter plate with 100 μL CAMHB per well. Untreated control and positive control (5% bleach) were also conducted. Bacterial inoculation (5×10⁵ CFU/mL) from an overnight culture was added to the plate (1 μL/well) and incubated at 37° C. for 20 hr. The change in optical density at 600 nm (ΔOD₆₀₀) was measured using a Tecan Infinite M20 plate reader immediately after inoculation Minimum inhibitory concentration (MIC) of each drug is determined as the lowest concentration that inhibited cell growth (ΔOD₆₀₀<0.05). Fractional inhibitory concentration index (FICI) and synergistic effects are determined using EUCAST guidelines: synergy (FICI≤0.5), additivity (0.5<FICI<1), and indifference (FICI>1). Each assay was done in triplicate.

Cell-Permeation Assay

bisBenzimide H33342 Intracellular Accumulation: Cryogenic stock of bacteria (MRSA OU6, OU11, MRSE 35984, or PA OU19) was inoculated overnight on tryptic soy agar. The culture was sub-inoculated in fresh tryptic soy broth (TSB) media for another 5-6 hours with shaking (100 rpm/min) at 37° C. Cells were pelleted by centrifugation at 7000 rpm for 40 min. The supernatant was discarded. The cells were resuspended in PBS and readjusted to OD₆₀₀=1.0. (which had a density of ˜7×10⁹ CFU/mL). Aliquots of the cell suspension were transferred to a 96-well flat-bottom black plate (180 μL/well) including the controls of the solvent (PBS blank), the untreated cells, and treated samples (either BPEI treated or polymyxin-B treated). Five technical replicates of each group were conducted. Hoechst 33342 bisbenzimide (H33342) was added (20 μL) to each well (final concentration of 5 μM). Fluorescence was read right after adding the H33342 by a Tecan Infinite M20 plate reader with the excitation and emission filters of 355 and 460 nm, respectively. Fluorescence data were normalized to the emission before cells were added in the PBS control (BPEI did not change the fluorescence by itself), and they were plotted against time to show the cellular uptake of H33342 over 10 minutes.

Cell Viability Assays

Cell viability assays with resazurin were performed with MRSA OU11 cells grown in TSB as similar to the procedure of cell-permeation assays until they reach a density of ˜7×10⁹ CFU/mL. Then the cell culture was transferred into a 96-well plate (100 μL/well) for BPEI or polymyxin B (PmB) treatments at varied concentrations from 64-512 μg/mL. Controls of untreated and positive control of 5% bleach were also conducted. The plate was incubated at 37° C. overnight. Resazurin (50 μL; final concentration of 50 μg/mL) was then added and, after 1 hour of incubation, the fluorescence intensity was measured (λₑₓ=560 nm; λₑₙ, =590 nm).

IL-8 Responses

HEKa cells were seeded in T-75 tissue culture flasks with Epilife media supplemented with human keratinocyte growth supplement 100 ug/mL and 100 U/mL of pen/strep and incubated at 37° C. in a CO₂ incubator. Fresh media was replaced every 2 days. Until the cell confluence reach 80-90%, they were split into a new passage. To avoid cell senescent, all experiments were performed with cells at passage 3-7. HEKa cells were cultured in a new 24-well plate until 80-90% confluence (total volume=1 mL/well). Then treatments of 600-Da BPEI (64, 128, 256, 512, and 1024 μg/mL) or S. aureus peptidoglycan (5 μg/mL) were added in triplicate cultures for 24 hr. The cell media was collected in 1.5 mL Eppendorf microtubes and stored at −20° C. until ELISA assays were performed. Concentrations of IL-8 cytokine released into the media were quantified followed the instructions of Quantikine Colorimetric ELISA assay kits (R&D Systems). Absorbance was measured at 450 nm and 570 nm. Final corrected absorbance was the subtraction at 450 nm from the one at 570 nm.

### Results

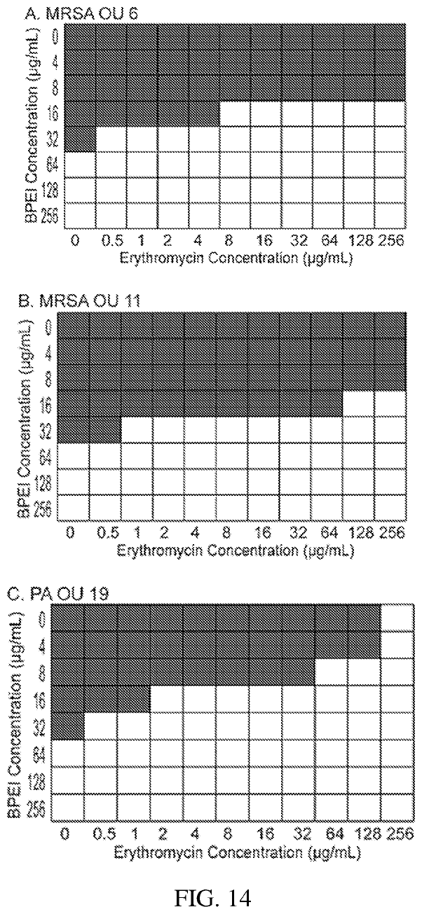

Erythromycin has long been used as a substitute for β-lactams for penicillin-allergic patients. It is a first-line treatment for many pediatric infections. Because erythromycin targets protein synthesis instead of the cell wall, it could be effective against methicillin-resistant staphylococci if the drug could reach the cytoplasm. The ability of 600-Da BPEI to increase erythromycin susceptibility was determined with in vitro checkerboard assays in 96-well microtiter plates. Two clinical MRSA isolates have erythromycin MICs over 2000 μg/mL. This demonstrates the strong resistance of MRSA. Because erythromycin targets intracellular targets, resistance could lie with hindered drug uptake imposed by the cell wall peptidoglycan and teichoic acids. This barrier to drug uptake has been described for polymyxins. However, 600-Da BPEI binds to these sites and this action improves the MRSA susceptibility to erythromycin. The MIC is 250 times lower in the presence of 16 μg/mL of 600-Da BPEI (FIG. 14 and Table 1). This broadens the spectrum of potential anti-MRSA drugs because, as previously reported, 600-Da BPEI was able to eliminate β-lactam resistance in these MRSA isolates and their biofilms. Against the MDR-PA clinical isolate OU19, 16 μg/mL BPEI lowers the erythromycin MIC from 256 to 2 μg/mL (FIG. 14 and Table 1). Moreover, a checkerboard assay demonstrated that a combination of azithromycin and 600-Da BPEI inhibits growth of Pseudomonas aeruginosa strain BAA-47. The MICs in these assays can be used to show synergy in the clinical isolate. These results demonstrates antibiotic potentiation against a formidable Gram-negative pathogen. Other macrolide antibiotics, in addition to erythromycin and azithromycin, that can be potentiated by being combined in treatment with BPEI or PEG-BPEI include, but not limited to, clarithromycin, fidaxomycin, and roxithromycin.

As shown in FIGS. 13A and 13B, potentiation by 600-Da BPEI relies on its interaction with different bacterial targets due to the different cell envelope architecture of MRSA and MDR-PA. Nevertheless, 600-Da BPEI can overcome both resistance barriers, and erythromycin potentiation by 600-Da BPEI is characterized as synergistic (Table 1). According to the EUCAST guidelines, a fractional inhibitory concentration index (FICI) is used to identify synergistic effects. FICI values can indicate synergy (FICI≤0.5), additivity (0.5<FICI<1), or indifference (FICI≥1). Erythromycin and 600-Da BPEI have synergy against MRSA (FICI=0.26 for OU6, 0.31 for OU11) and MDR-PA OU19 (FICI=0.26).

Without wishing to be bound by theory, synergy between 600-Da BPEI and erythromycin can be attributed to increased drug influx. Resistance to non-β-lactam antibiotics (such as macrolides, tetracyclines, and fluoroquinolones) involves membrane-bound efflux-pump proteins. These protein assemblies expel toxic substances (e.g., antibiotics), hindering accumulation of antibiotics in bacterial cells. To examine increased drug influx, we tested the ability of 600-Da BPEI to increase the intracellular concentration of a fluorescence probe molecule, Hoechst 33342 bisbenzimide (H33342). H33342 fluorescence increases when it penetrates the cell-membrane and binds to intracellular DNA. Greater accumulation of H33342 in the cells creates a higher fluorescence intensity. Fluorescence measurements were taken for untreated and BPEI-treated samples immediately after adding the H33342 dye.

TABLE 1
Synergy of 600-Da BPEI and antibiotics against
MRSA and MDR-PA clinical isolates

| | | | | MIC^{[a]} | | |
|---|---|---|---|---|---|---|
| | | | | Antibiotic | | |
| | | MIC^{[a]} | MIC^{[a]} | with 16 | | |
| | Anti- | BPEI | Antibiotic | μg/ml | | |
| Strain | biotic | (μg/mL) | (μg/mL) | BPEI | FICI^{[b]} | Outcome |
| MRSA | Erythro- | 64 | >2000 | 8 | 0.26 | Synergy |
| OU6 | mycin | | | | | |
| MRSA | Erythro- | 64 | >2000 | 128 | 0.31 | Synergy |
| OU11 | mycin | | | | | |
| PA | Erythro- | 64 | 256 | 2 | 0.26 | Synergy |
| OU19 | mycin | | | | | |

^{[a]}MIC, Minimum Inhibitory Concentration;
^{[b]}FICI, the Minimum Fractional Inhibitory Concentration Index

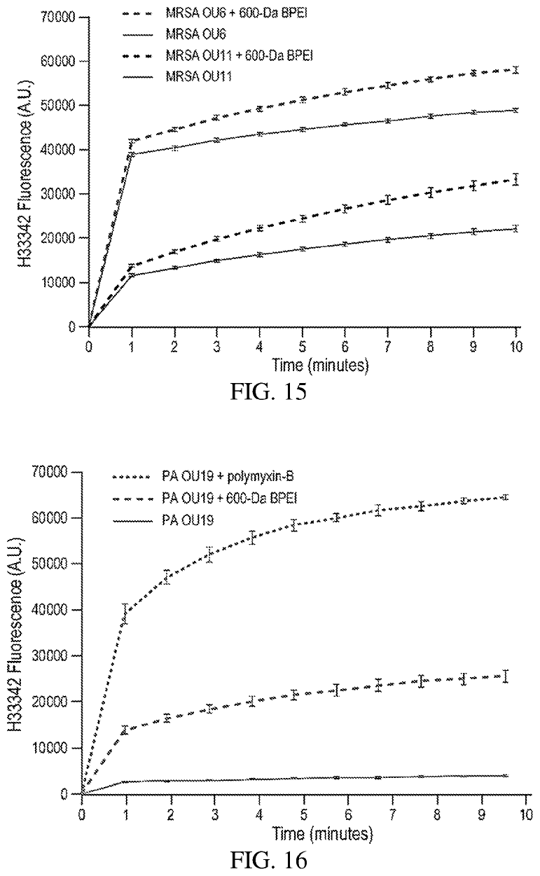

As shown in FIGS. 15 and 17, 600-Da BPEI enhanced dye uptake in MRSA OU6 and MRSA OU11. The BPEI-treated cells had much higher fluorescence intensity by approximately 10,000 fluorescence units compared to their untreated controls. Similar trends are observed for the influx of H33342 into cells of the MDR-PA clinical isolate OU19 (FIG. 16). P. aeruginosa is well-known for a powerful drug-efflux system and thus the low accumulation of H33342, compared to Gram-positive MRSA, is not unexpected. Nonetheless, as with MRSA, 600-Da BPEI increases the uptake of H33342 in the MDR-PA OU19.

The concentration of BPEI used in fluorescence assays, 128 μg/mL, would appear to be a lethal concentration as it is greater than the MIC of each isolate. However, an important consideration is that generating fluorescent signals above the detection limit requires a higher cell density (˜7×10⁹ CFU/mL) than in checkerboard assays (˜5×10⁵ CFU/mL). Thus, while 128 μg/ml of 600-Da BPEI is lethal in the checkerboard assays, it is sub-lethal in the fluorescence studies. This is shown by measuring cell viability using a resazurin cellular metabolism assay. MRSA OU11 was grown until it reached the same cell density in H33342 assays (˜7×10⁹ CFU/mL) before BPEI treatment. Resazurin was then added and, after 1 hour of incubation, the fluorescence intensity was measured. Cellular metabolic product NADH irreversibly reduces resazurin into resorufin, which emits strong fluorescence at 580-590 nm, indicating cell viability. As shown in FIG. 18, MRSA cells were slightly less viable with higher concentrations of 600-Da BPEI, suggesting that the membrane remains intact. Previous studies showed that BPEI attached to the surface of MRSA cells. Additionally, scanning electron micrography of MRSA show that sub-lethal amount of 600-Da BPEI altered the cell wall morphology. These data support an MOA involving the ability of 600-Da BPEI to weaken the cell envelope rather than lysing the bacteria. This is a different MOA than that of polymyxin-B, a cationic antibiotic known to cause widespread disruption of bacterial membranes.

Using the resazurin assay, corresponding concentrations of polymyxin-B were more lethal to MRSA OU11 (FIG. 18) than 600-Da BPEI. In fact, 64 μg/mL of polymyxin-B (PmB) caused greater cell death than 512 μg/mL 600-Da BPEI, and the highest concentration of PmB (512 μg/mL) resulted in complete loss of MRSA OU11 viability. These experiments highlight the low antibiotic propensity, but high potentiation ability, of 600-Da BPEI. These data also support the paradigm that PmB is considered a Gram-negative selective drug due to its low MICs (≤2 μg/mL), while Gram-positive bacteria require much higher concentration of PmB (≥32 μg/mL) due to the diffusion barrier imposed by the thick bacterial cell wall.

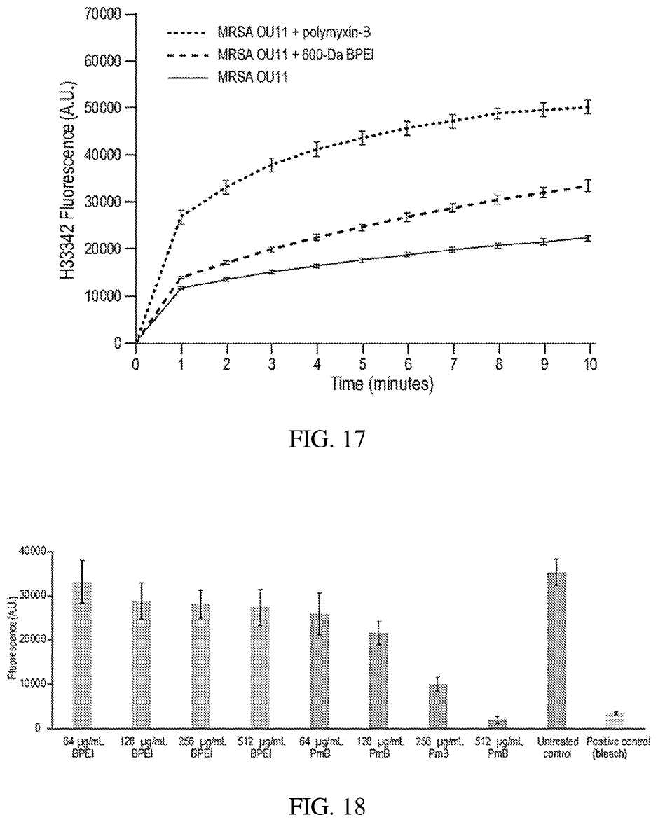

The different MOAs of 600-Da BPEI and PmB can be examined by gauging their effect on the influx of H33342. The clinical isolate PA OU19 (FIG. 16) and MRSA OU11 (FIG. 17) were exposed to similar amounts of 600-Da BPEI and PmB. As shown, PmB dramatically increased the intracellular concentration of H33342 by disrupting the membrane bilayer using its hydrophobic alkyl tail. In contrast, 600-Da BPEI is hydrophilic and lacks the energetic driving force to penetrate the membrane. Thus, BPEI reduces drug diffusion barriers within the peptidoglycan layer of MRSA and LPS of PA without damaging the membrane. This MOA also explains why the rate of H33342 influx during the first few minutes is much higher for PmB than 600-Da BPEI. The ability of PmB to disrupt membrane layers aligns with the literature precedent that PmB is nephrotoxic and neurotoxic towards human cells. In contrast, 600-Da BPEI is unlikely to damage the membranes but instead reduces drug-influx barriers that allows faster diffusion through the bacterial membrane that allows enhanced H33342 accumulation in the MRSA and PA cells.

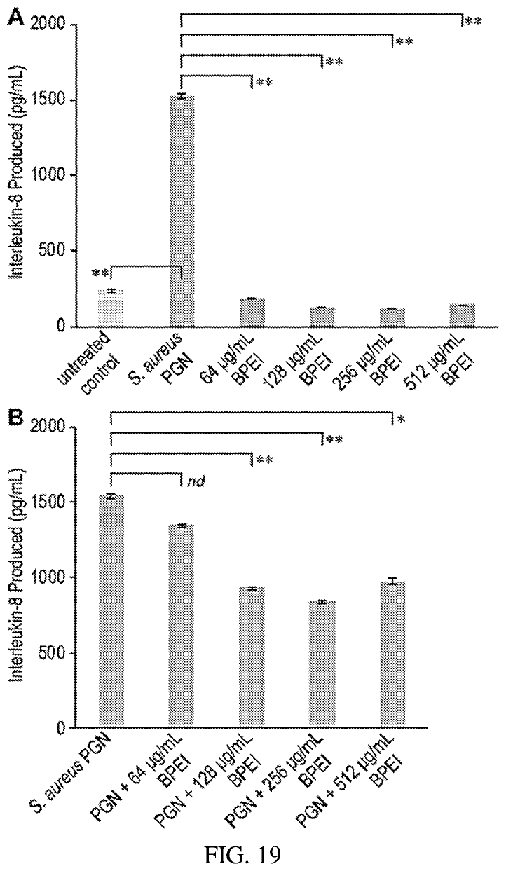

Staphylococci are notorious for their skin and soft-tissue infections that often lead to more complicated diseases. Each year, millions of acute SSTIs become chronic wound infections. Instead of taking 3-6 weeks to heal, chronic wounds persist for 3-6 months. Delays in healing acute SSTIs are often due to a prolonged inflammatory phase of healing caused by bacterial debris, such as peptidoglycan from S. aureus, which is a PAMP molecule. Preventing S. aureus peptidoglycan from triggering the release of inflammatory cytokines will restore the optimal inflammatory response. However, successful drugs are elusive because the cell wall debris has a large variation in size and shape, making it virtually impossible to target peptidoglycan with monoclonal antibodies that recognize specific polysaccharide units. Instead, 600-Da BPEI binds the anionic sites of peptidoglycan and inhibits its ability to stimulate cytokine production. As shown in FIG. 19 S. aureus peptidoglycan promotes the release of interleukin-8 (IL-8) from primary human epithelial keratinocytes (HEKa) cells. IL-8 is a cytokine and chemokine molecule involved with neutrophil recruitment to the wound site. Its production is stimulated when peptidoglycan binds to, and is mainly recognized by, toll-like receptor 2 (TLR2). In contrast, 600-Da BPEI does not promote IL-8 production by the HEKa cells (FIG. 19(A)). However, when 600-Da BPEI and S. aureus peptidoglycan are added to HEKa cells, the amount of IL-8 diminishes (FIG. 19(B)), indicating another promising therapeutic benefit of BPEI for wound care.

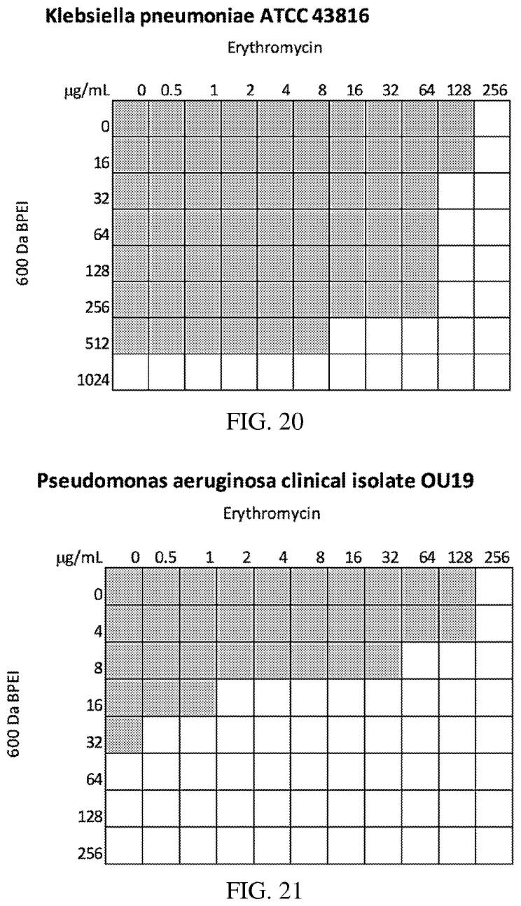

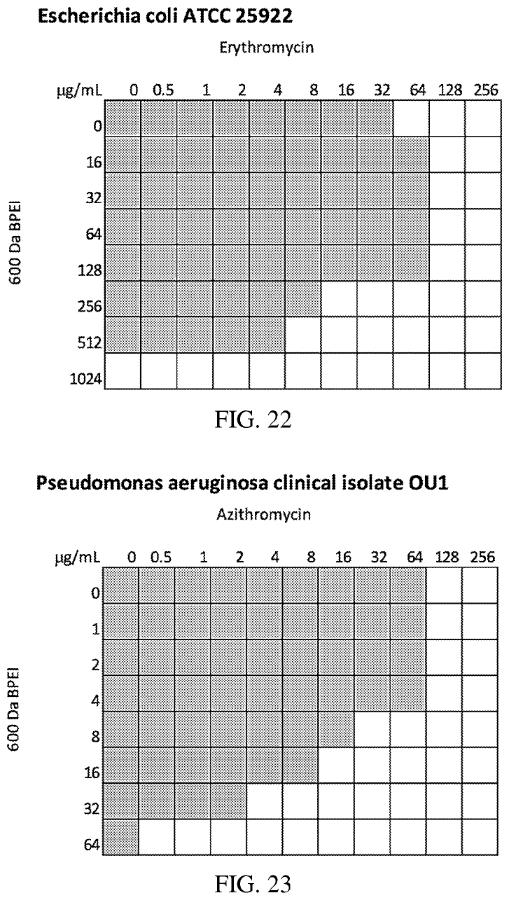

FIGS. 20-22 show inhibition of growth of strains of K. pneumoniae, P. aeruginosa, and E. coli by combinations of various concentrations of erythromycin and 600-Da BPEI. The MICs in these assays can be used to show synergy of erythromycin and 600-Da BPEI against the strains. Synergy between 600-Da BPEI and erythromycin is attributed to increased drug influx. Resistance to non-β-lactam antibiotics (such as macrolides) involves membrane-bound efflux-pump proteins. These protein assemblies expel toxic substances (e.g. antibiotics), hindering accumulation of antibiotics in bacterial cells.

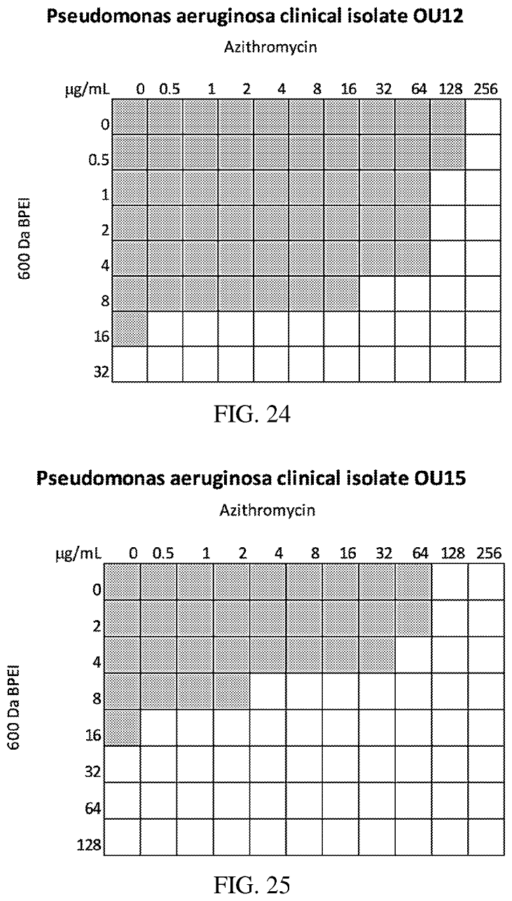

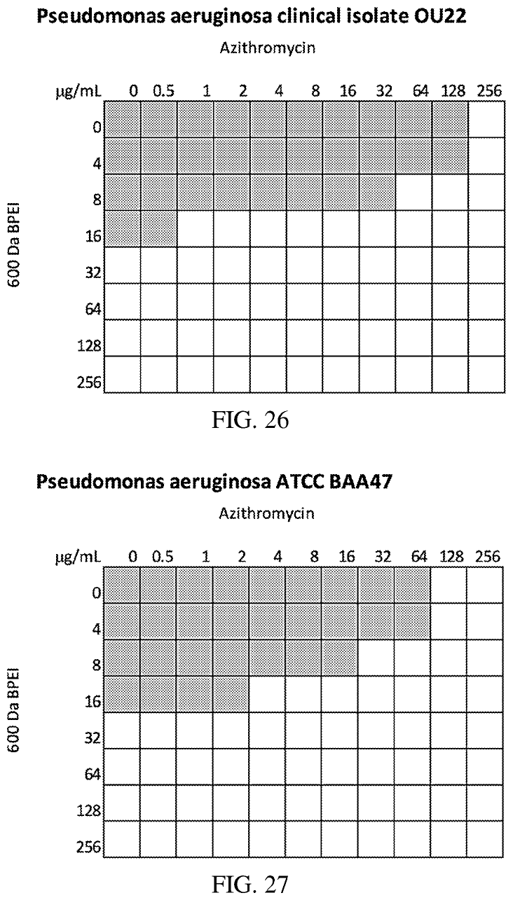

FIGS. 23-27 show inhibition of growth of several P. aeruginosa by clinical isolates and an ATCC strain by combinations of various concentrations of azithromycin\ and 600-Da BPEI. The MICs in these assays can be used to show synergy of azithromycin and 600-Da BPEI against the strains. P. aeruginosa is well-known for a powerful drug-efflux system and thus overcoming drug efflux processes increases the impact of 600-Da BPEI potentiatiors. These data demonstrate the activity of 600-Da BPEI against multidrug-resistant P. aeruginosa which can be used for developing broad-spectrum antibiotic and potentiator combinations. Antibiotic combination therapy using existing drugs also reserves the newer last-resort antibiotics for use against the most serious cases of antibiotic-resistant infection.

As demonstrated above, BPEI or PEG-BPEI can be used in combination with macrolide antibiotics such as erythromycin and azithromycin to potentiate the effects of the antibiotics against both Gram-positive and Gram-negative bacteria.

In summary, therefore, in at least certain non-limiting embodiments, the present disclosure includes a method of treating an inflammatory condition in a subject in need of such therapy, comprising: administering a branched poly(ethylenimine) (BPEI) compound to an epithelium of the subject, the BPEI compound administered in a quantity sufficient to inhibit release of at least one cytokine from epithelial cells of the epithelium, wherein the at least one cytokine is selected from the group consisting of tumor necrosis factor alpha (TNFα), interleukin-6 interleukin-8 (IL-8), and a Type-1 interferon. The epithelial cells may be, for example, human epithelial keratinocytes, human lung epithelial cells, or human intestinal epithelial cells. In any of the above embodiments, the BPEI molecule may have an average Mw in a range of about 0.1 kilodalton (kDa) to about 25 kDa. In any of the above embodiments, the BPEI molecule may have an average Mw of about 600 Da. In any of the above embodiments, the BPEI compound may comprise a polyethylene glycol (PEG) molecule conjugated to the BPEI molecule forming a PEG-BPEI conjugate. In any of the above embodiments, the inflammatory condition may occur in a wound. In any of the above embodiments, the wound may be an acute wound or a chronic wound. In any of the above embodiments, the inflammatory condition may occurs in a lung. In any of the above embodiments, the BPEI compound may be conjointly administered with an antibiotic, wherein the BPEI compound and antibiotic, when administered conjointly, are effective in inhibiting a bacterium. In any of the above embodiments, the bacterium may be one or more of Staphylococcus aureus, methicillin-resistant Staphylococcus aureus (MRSA), oxacillin-resistant Staphylococcus aureus (ORSA), vancomycin-resistant Staphylococcus aureus (VRSA), Staphylococcus epidermidis, methicillin-resistant Staphylococcus epidermidis (MRSE), Enterococcus faecalis, Enterococcus faecium, a Streptococcus pneumoniae, penicillin-resistant Streptococcus pneumoniae, Streptococcus mutans, Streptococcus sanguinis, Streptococcus viridans, Bacillus anthracis, Bacillus cereus, Clostridium botulinum, Listeria monocytogenes, Klebsiella pneumoniae, Klebsiella oxytoca, Klebsiella sp., Escherichia coli, pathogenic Escherichia coli, Pseudomonas aeruginosa, multidrug-resistant Pseudomonas aeruginosa, Salmonella enterica, Salmonella sp., and Shigella sp. In any of the above embodiments, the BPEI compound may be provided in a composition comprising a carrier or vehicle selected from the group consisting of ointments, creams, pastes, gums, lotions, gels, foams, emulsions, suspensions, aqueous solutions, powders, lyophilized powders, solutions, granules, foams, drops, eye drops, adhesives, sutures, aerosols, sprays, sticks, soaps, bars of soap, balms, body washes, rinses, tinctures, gel beads, gauzes, wound dressings, bandages, cloths, towelettes, stents, and sponges. In any of the above embodiments, the BPEI compound may bind to a bacterium-derived product selected from the group consisting of lipopolysaccharide (LPS), lipoteichoic acid (LTA), and peptidoglycan (PG). In any of the above embodiments, the Type-1 interferon may be selected from IFN-α, IFN-β and IFN-γ. In any of the above embodiments, the method may comprise treating an inflammatory condition in a subject in need of such treatment, the inflammatory condition comprising a chronic or ischemic site of inflammation, the method comprising: topically applying or injecting to the site of inflammation, for a therapeutically effective period of time, a therapeutically effective amount of an anti-inflammatory conjugate comprising a branched poly(ethylenimine) (BPEI) molecule conjugated to a polyethylene glycol (PEG) molecule. The site of inflammation may be a site of dermal inflammation or a wound. The wound may be selected from the group consisting of acute wounds and chronic wounds.

In further summary, in at least certain non-limiting embodiments, the present disclosure includes a method of inhibiting a carbapenem-resistant Enterobacteriaceae (CRE) bacterium, comprising conjointly administering to the CRE bacterium a carbapenem antibiotic, and a branched poly(ethylenimine) (BPEI) compound comprising a BPEI molecule, and optionally a β-lactamase inhibitor, wherein the carbapenem antibiotic and the BPEI compound, and optionally the β-lactamase inhibitor, are conjointly administered in amounts sufficient to potentiate the inhibitory activity of the carbapenem antibiotic against the CRE bacterium, wherein the carbapenem antibiotic is provided in an amount sufficient to reduce the minimum inhibitory concentration (MIC) of the carbapenem antibiotic below a resistence breakpoint thereof for the CRE bacterium. In any of the above embodiments, the BPEI molecule may have an average Mw in a range of about 0.1 kilodalton (kDa) to about 25 kDa. In any of the above embodiments, the BPEI molecule may have an average Mw of about 600 Da. In any of the above embodiments, the BPEI molecule of the BPEI compound may be conjugated to a polyethylene glycol (PEG) molecule forming a PEG-BPEI conjugate. In any of the above embodiments, the CRE bacterium may be one or more of Escherichia coli, Klebsiella pneumoniae, Klebsiella oxytoca, Enterobacter aerogenes, Enterobacter cloacae, Enterobacter gergoviae, Citrobacter freundii, Citrobacter koseri, Salmonella enterica, Proteus mirabilis, Serratia marcescens, Morganella morganii, Providencia stuartii, and Providencia rettgeri. In any of the above embodiments, the carbapenem antibiotic may be one or more of meropenem, imipenem, ertapenem, doripenem, biapenem, faropenem, tebipenem, and panipenem. In any of the above embodiments, the β-lactamase inhibitor may be selected from the group consisting of vaborbactam, tazobactam, clavulanate, sulbactam, and diazobicyclooctanes. In any of the above embodiments, the diazobicyclooctane may be selected from the group consisting of avibactam and relebactam. In any of the above embodiments, the carbapenem antibiotic and the BPEI compound together may have a synergistic fractional inhibitory concentration (FIC) against the CRE bacterium, wherein the FIC≤0.5. In any of the above embodiments, the CRE bacterium may be inhibited in a subject having a CRE bacterial infection. In any of the above embodiments, the subject may be a human or a non-human animal. In any of the above embodiments, the bacterial infection may be an acute or chronic wound in the subject. In any of the above embodiments, the BPEI compound may be provided in a composition comprising a carrier or vehicle selected from the group consisting of ointments, creams, pastes, gums, lotions, gels, foams, emulsions, suspensions, aqueous solutions, powders, lyophilized powders, solutions, granules, foams, drops, eye drops, adhesives, sutures, aerosols, sprays, sticks, soaps, bars of soap, balms, body washes, rinses, tinctures, gel beads, gauzes, wound dressings, bandages, cloths, towelettes, stents, and sponges.

In further summary, in at least certain non-limiting embodiments, the present disclosure includes an antibiotic composition, comprising a carbapenem antibiotic, and a branched poly(ethylenimine) (BPEI) compound comprising a BPEI molecule, and optionally a β-lactamase inhibitor, in amounts which when administered conjointly to a carbapenem-resistant Enterobacteriaceae (CRE) bacterium cause the inhibitory effect of the carbapenem antibiotic against the CRE bacterium to be potentiated to a degree that the carbapenem antibiotic reduces the minimum inhibitory concentration (MIC) of the carbapenem antibiotic to a level below a resistence breakpoint for the CRE bacterium. In any of the above embodiments, the BPEI molecule may have an average Mw in a range of about 0.1 kilodalton (kDa) to about 25 kDa. In any of the above embodiments, the BPEI molecule may have an average Mw of about 600 Da. In any of the above embodiments, the the BPEI molecule of the BPEI compound may be conjugated to a polyethylene glycol (PEG) molecule forming a PEG-BPEI conjugate. In any of the above embodiments, the CRE bacterium may be one or more of Escherichia coli, Klebsiella pneumoniae, Klebsiella oxytoca, Enterobacter aerogenes, Enterobacter cloacae, Enterobacter gergoviae, Citrobacter freundii, Citrobacter koseri, Salmonella enterica, Proteus mirabilis, Serratia marcescens, Morganella morganii, Providencia stuartii, and Providencia rettgeri. In any of the above embodiments, the carbapenem antibiotic may be one or more of meropenem, imipenem, ertapenem, doripenem, biapenem, faropenem, tebipenem, and panipenem. In any of the above embodiments, the β-lactamase inhibitor may be one or more of vaborbactam, tazobactam, clavulanate, sulbactam, and diazobicyclooctanes. In any of the above embodiments, the diazobicyclooctane may be selected from the group consisting of avibactam and relebactam. In any of the above embodiments, the carbapenem antibiotic and the BPEI compound together have a synergistic fractional inhibitory concentration (FIC) against the CRE bacterium, wherein the FIC≤0.5. In further summary, in at least certain non-limiting embodiments, the present disclosure includes a kit, comprising a first container which contains a carbapenem antibiotic, and a second container which contains a potentiating compound comprising a branched poly(ethylenimine) (BPEI) molecule, wherein the carbapenem antibiotic and the potentiating compound have synergistic activity against a carbapenem-resistant Enterobacteriaceae (CRE) when administered conjointly. In any of the above embodiments, the BPEI molecule may be conjugated to a polyethylene glycol (PEG) molecule forming a PEG-BPEI conjugate. In any of the above embodiments, the kit may further comprise a β-lactamase inhibitor.

In further summary, in at least certain non-limiting embodiments, the present disclosure includes a method of treating a bacterial infection in a subject in need of such treatment, comprising conjointly administering to the subject a macrolide antibiotic, and a potentiating compound comprising a branched poly(ethylenimine) (BPEI) molecule, wherein the macrolide antibiotic and the potentiating compound are administered in amounts sufficient to have synergistic activity against the bacterium when administered conjointly. In any of the above embodiments, the BPEI molecule may have an average Mw in a range of about 0.1 kilodalton (kDa) to about 25 kDa. In any of the above embodiments, the macrolide antibiotic may be one or more of erythromycin, azithromycin, clarithromycin, fidaxomycin, and roxithromycin. In any of the above embodiments, the BPEI molecule may have an average Mw of about 600 Da. In any of the above embodiments, the BPEI molecule of the potentiating compound may be conjugated to a polyethylene glycol (PEG) molecule forming a PEG-BPEI conjugate. In any of the above embodiments, the bacterium may be one or more of Staphylococcus aureus, methicillin-resistant Staphylococcus aureus (MRSA), oxacillin-resistant Staphylococcus aureus (ORSA), vancomycin-resistant Staphylococcus aureus (VRSA), Staphylococcus epidermidis, methicillin-resistant Staphylococcus epidermidis (MRSE), Enterococcus faecalis, Enterococcus faecium, a Streptococcus pneumoniae, penicillin-resistant Streptococcus pneumoniae, Streptococcus mutans, Streptococcus sanguinis, Streptococcus viridans, Bacillus anthracis, Bacillus cereus, Clostridium botulinum, Listeria monocytogenes, Klebsiella pneumoniae, Klebsiella oxytoca, Klebsiella sp., Escherichia coli, pathogenic Escherichia coli, Pseudomonas aeruginosa, multidrug-resistant P. aeruginosa, Salmonella enterica, Salmonella sp., and Shigella sp. In any of the above embodiments, the macrolide antibiotic and the BPEI compound together have a synergistic fractional inhibitory concentration (FIC) against the bacterium, wherein the FIC≤0.5. In any of the above embodiments, the bacterial infection may be an acute or chronic wound in the subject. In any of the above embodiments, the potentiating compound may be provided in a composition comprising a carrier or vehicle selected from the group consisting of ointments, creams, pastes, gums, lotions, gels, foams, emulsions, suspensions, aqueous solutions, powders, lyophilized powders, solutions, granules, foams, drops, eye drops, adhesives, sutures, aerosols, sprays, sticks, soaps, bars of soap, balms, body washes, rinses, tinctures, gel beads, gauzes, wound dressings, bandages, cloths, towelettes, stents, and sponges. In any of the above embodiments, the treatment may result in a reduction in the release of at least one cytokine from a tissue to which the potentiating compound and antibiotic is administered, wherein the cytokine is selected from the group consisting of tumor necrosis factor alpha (TIS Fa), interleukin-6 interleukin-8 (IL-8), and a Type-1 interferon.

In further summary, in at least certain non-limiting embodiments, the present disclosure includes a method of treating a subject for an inflammatory condition, the inflammatory condition comprising a chronic or ischemic site of inflammation in the subject, the method comprising conjointly topically applying or injecting, for a therapeutically effective period of time, a therapeutically effective amount of a macrolide antibiotic and an anti-inflammatory potentiating compound comprising a branched poly(ethylenimine) (BPEI) molecule to the site of inflammation, thereby reducing the inflammatory condition in the subject. In any of the above embodiments, the BPEI molecule may have an average Mw in a range of about 0.1 kilodalton (kDa) to about 25 kDa. In any of the above embodiments, the BPEI molecule may have an average Mw of about 600 Da. In any of the above embodiments, the BPEI molecule of the potentiating compound may be conjugated to a polyethylene glycol (PEG) molecule forming a PEG-BPEI conjugate. In any of the above embodiments, the macrolide antibiotic may be one or more of erythromycin, azithromycin, clarithromycin, fidaxomycin, and roxithromycin. In any of the above embodiments, the site of inflammation may be a site of dermal inflammation. In any of the above embodiments, the site of inflammation may be an acute or chronic wound in the subject. In further summary, in at least certain non-limiting embodiments, the present disclosure includes a kit comprising a first container which contains a macrolide antibiotic, and a second container which contains a potentiating compound comprising a branched poly(ethylenimine) (BPEI) molecule, wherein the macrolide antibiotic and the potentiating compound have synergistic activity against a bacterium when administered conjointly. In any of the above embodiments, the BPEI molecule of the potentiating compound may be conjugated to a polyethylene glycol (PEG) molecule forming a PEG-BPEI conjugate. In any of the above embodiments, the bacterium may be one or more of Staphylococcus aureus, methicillin-resistant Staphylococcus aureus (MRSA), oxacillin-resistant Staphylococcus aureus (ORSA), vancomycin-resistant Staphylococcus aureus (VRSA), Staphylococcus epidermidis, methicillin-resistant Staphylococcus epidermidis (MRSE), Enterococcus faecalis, Enterococcus faecium, a Streptococcus pneumoniae, penicillin-resistant Streptococcus pneumoniae, Streptococcus mutans, Streptococcus sanguinis, Streptococcus viridans, Bacillus anthracis, Bacillus cereus, Clostridium botulinum, Listeria monocytogenes, Klebsiella pneumoniae, Klebsiella oxytoca, Klebsiella sp., Escherichia coli, pathogenic Escherichia coli, Pseudomonas aeruginosa, multidrug-resistant Pseudomonas aeruginosa, Salmonella enterica, Salmonella sp., and Shigella sp.

While the present disclosure has been described in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the presently disclosed methods and compositions. Changes may be made in the compositions and methods described herein, or in the steps or the sequence of steps of the methods described herein, without departing from the spirit and scope of the present disclosure.

1. A method of treating an inflammatory condition in a subject in need of such therapy, comprising: administering a branched poly(ethylenimine) (BPEI) compound to an epithelium of the subject, the BPEI compound inhibiting the release of at least one cytokine from epithelial cells of the epithelium, wherein the at least one cytokine is selected from the group consisting of tumor necrosis factor alpha (TNFα), interleukin-6 (IL-6), interleukin-8 (IL-8), and a Type-1 interferon.

2. The method of claim 1, wherein the epithelial cells are selected from the group consisting of human epithelial keratinocytes, human lung epithelial cells, and human intestinal epithelial cells.

3. The method of claim 1, wherein the BPEI molecule has an average Mw in a range of about 0.1 kilodalton (kDa) to about 25 kDa.

4. The method of claim 1, wherein the BPEI molecule has an average Mw of about 600 Da.

5. The method of claim 1, wherein the BPEI compound comprises a polyethylene glycol (PEG) molecule conjugated to the BPEI molecule forming a PEG-BPEI conjugate.

6. The method of claim 1, wherein the inflammatory condition occurs in a wound.

7. The method of claim 6, wherein the wound is an acute wound.

8. The method of claim 6, wherein the wound is a chronic wound.

9. The method of claim 1, wherein the inflammatory condition occurs in a lung.

10. The method of claim 1, wherein the BPEI compound is conjointly administered with an antibiotic, wherein the BPEI compound and antibiotic, when administered conjointly, are effective in inhibiting a bacterium.

11. The method of claim 10, wherein the bacterium is selected from the group consisting of Staphylococcus aureus, methicillin-resistant Staphylococcus aureus (MRSA), oxacillin-resistant Staphylococcus aureus (ORSA), vancomycin-resistant Staphylococcus aureus (VRSA), Staphylococcus epidermidis, methicillin-resistant Staphylococcus epidermidis (MRSE), Enterococcus faecalis, Enterococcus faecium, a Streptococcus pneumoniae, penicillin-resistant Streptococcus pneumoniae, Streptococcus mutans, Streptococcus sanguinis, Streptococcus viridans, Bacillus anthracis, Bacillus cereus, Clostridium botulinum, Listeria monocytogenes, Klebsiella pneumoniae, Klebsiella oxytoca, Klebsiella sp., Escherichia coli, pathogenic Escherichia coli, Pseudomonas aeruginosa, multidrug-resistant Pseudomonas aeruginosa, Salmonella enterica, Salmonella sp., and Shigella sp.

12. The method of claim 1, wherein the BPEI compound is provided in a composition comprising a carrier or vehicle selected from the group consisting of ointments, creams, pastes, gums, lotions, gels, foams, emulsions, suspensions, aqueous solutions, powders, lyophilized powders, solutions, granules, foams, drops, eye drops, adhesives, sutures, aerosols, sprays, sticks, soaps, bars of soap, balms, body washes, rinses, tinctures, gel beads, gauzes, wound dressings, bandages, cloths, towelettes, stents, and sponges.

13. The method of claim 1, wherein the BPEI compound binds to a bacterium-derived product selected from the group consisting of lipopolysaccharide (LPS), lipoteichoic acid (LTA), and peptidoglycan (PG).

14. The method of claim 1, wherein the Type-1 interferon is selected from IFN-α, IFN-β and IFN-γ.

15. A method of treating an inflammatory condition in a subject in need of such treatment, the inflammatory condition comprising a chronic or ischemic site of inflammation, the method comprising: topically applying or injecting to the site of inflammation, for a therapeutically effective period of time, a therapeutically effective amount of an anti-inflammatory conjugate comprising a branched poly(ethylenimine) (BPEI) molecule conjugated to a polyethylene glycol (PEG) molecule.

16. The method of claim 15, wherein the site of inflammation is a site of dermal inflammation or a wound.

17. The method of claim 16, wherein the wound is selected from the group consisting of acute wounds and chronic wounds.

18-66. (canceled)

67. The method of claim 1, further comprising administering to the subject a macrolide antibiotic conjointly with the BPEI.

68. The method of claim 67, wherein the macrolide antibiotic is selected from the group consisting of erythromycin, azithromycin, clarithromycin, fidaxomycin, and roxithromycin.

69. The method of claim 15, further comprising administering to the subject a macrolide antibiotic conjointly with the anti-inflammatory conjugate.

70. The method of claim 69, wherein the macrolide antibiotic is selected from the group consisting of erythromycin, azithromycin, clarithromycin, fidaxomycin, and roxithromycin.
